(19) United States
(12) Patent Application Publication
(10) Pub. No.: US 2023/0119558 A1
(43) Pub. Date: Apr. 20, 2023

(54) DNA DAMAGE REPAIR GENES IN CANCER

(71) Applicant: The Henry M. Jackson Foundation for the Advancement of Military Medicine, Inc., Bethesda, MD (US)

(72) Inventors: Gyorgy PETROVICS, Bethesda, MD (US); Shiv K. SRIVASTAVA, Potomac, MD (US)

(21) Appl. No.: 17/909,500

(22) PCT Filed: Mar. 5, 2021
(86) PCT No.: PCT/US2021/021136

§ 371 Date: Sep. 6, 2022

Related U.S. Application Data
(60) Provisional application No. 62/985,996, filed on Mar. 6, 2020.

(51) Int. Cl. C12Q 1/6886

(52) U.S. Cl. CPC C12Q 1/6886; C12Q 1/6858

(57) ABSTRACT

The present disclosure provides DNA Damage Repair gene (DDRG) panels and methods of using the same for genetic testing and genetic counseling to predict a predisposition to cancer, including prostate cancer. The gene panels can be used to stratify prostate cancer patients according to disease severity and/or aggressiveness or to identify and/or stratify a patient for cancer treatment. Also provided are kits for use in predicting, diagnosing, and/or prognosing cancer.

### CROSS REFERENCE TO RELATED APPLICATION

This application claims the benefit of, and relies on the filing date of, U.S. Provisional Pat. Application No. 62/985,996, filed 6 Mar. 2020, the entire disclosure of which is incorporated herein by reference.

### GOVERNMENT INTEREST

This invention was made with government support under grant number DHA-16-CRII-PMRA, awarded by the Department of Defense. The government has certain rights in the invention.

### FIELD

This application relates generally to DNA damage repair genes (DDRG) panels, and more specifically to the use of DDRG or panels comprising the same for predicting, diagnosing, and prognosing cancer, such as prostate cancer, particularly in patients having a history of cancer or in patients of various ethnicities, such as prostate cancer in patients of African descent and prostate cancer in patients of Caucasian decent.

### BACKGROUND

Cancer is a leading cause of death worldwide, with the United States having an estimated more than 1,700,000 new cancer diagnoses and over 600,000 cancer fatalities in a single year. In particular, prostate cancer is the second leading cause of cancer death among men in the United States, with an anticipated 174,650 newly diagnosed cases and approximately 31,620 deaths in 2019. Siegel, R. et al., Cancer statistics, CA Cancer J. Clin. 2019; 69:7-34.

It is estimated that 1 in 6 men of African ancestry will be diagnosed with cancer of the prostate (CaP) in their lifetime, in comparison with 1 in 8 men of Caucasian ancestry. Emerging data support biological and genetic differences between African-American (AA) and Caucasian-American (CA) CaP. The racial disparity exists from presentation and diagnosis through treatment, survival, and quality of life. Chornokur, G. et al., Disparities at presentation, diagnosis, treatment, and survival in African American men affected by prostate cancer, Prostate 2011; 71:985-997. Researchers have suggested that socio-economic status (SES) contributes significantly to these disparities, including CaP-specific mortality. Schwartz, K. et al., Interplay of race, socioeconomic status, and treatment on survival of patients with prostate cancer, Urology 2009; 74:1296-1302. There is also evidence that reduced access to care is associated with poor CaP outcomes, which is more prevalent among men of AD than men of CD. Major, J.M. et al., Socioeconomic status, healthcare density, and risk of prostate cancer among African American and Caucasian men in a large prospective study, Cancer Causes Control 2012; 23:1185-1191.

However, there are populations in which men of AD have similar outcomes to men of CD. Sridhar and colleagues published a meta-analysis in which they concluded that when SES is accounted for, there are no differences in the overall and CaP-specific survival between men of CD and AD. Sridhar, G. et al., Do African American men have lower survival from prostate cancer compared with White men? A meta-analysis, Am. J Men’s Health 2010, 4:189-206. Similarly, the military and veteran populations (systems of equal access and screening) do not observe differences in survival across race, and differences in pathologic stage at diagnosis narrowed by the early 2000s in a veterans’ cohort. Cullen, J. et al., Racial/ethnic patterns in prostate cancer outcomes in an active surveillance cohort, Prostate Cancer 2011; doi:10.1155/2011/234519; and Berger, A.D. et al., Differences in clinicopathologic features of prostate cancer between black and white patients treated in the 1990s and 2000s, Urology 2006; 67:120-124. Of note, both Cullen et al. and Berger et al. showed that men of AD were more likely to have higher Gleason scores and prostate-specific antigen (PSA) levels than men of CD.

While socio-economic factors may contribute to CaP outcomes, they do not seem to account for all variables associated with the diagnosis and disease risk. Several studies support that men of AD have a higher incidence of CaP compared to men of CD. Siegel et al, 2019; Kheirandish, P. et al., Ethnic differences in prostate cancer, Br. J. Cancer 2011; 105:481-485; and Odedina, F.T. et al., Prostate cancer disparities in black men of African descent: A comparative literature review of prostate cancer burden among black men in the United States, Caribbean, United Kingdom, and West Africa, Infect. Agents Cancer 2009; 4: doi:10.1186/1750-9378-4S1-S2. Studies also show that men of AD have a significantly higher PSA at diagnosis, higher grade disease on biopsy, greater tumor volume for each stage, and a shorter PSA doubling time before radical prostatectomy. Heath, E.I. et al., The effect of race/ethnicity on the accuracy of the 2001 Partin Tables for predicting pathologic stage of localized prostate cancer, Urology 2008; 71:151-155; Moul, J.W. et al., Prostate-specific antigen values at the time of prostate cancer diagnosis in African-American men, JAMA 1995; 274:1277-1281; and Tewari, A. et al., Racial differences in serum prostate-specific (PSA) doubling time, histopathological variables and long-term PSA recurrence between African-American and white American men undergoing radical prostatectomy for clinically localized prostate cancer, BJU Int. 2005; 96:29-33. Biological differences between prostate cancers from men of CD and AD have been noted in the tumor microenvironment with regard to stress and inflammatory responses. Wallace, T.A. et al., Tumor immunobiological differences in prostate cancer between African-American and Caucasian-American men, Cancer Res. 2008; 68:927-936. Although questions remain to be clarified over the role of biological differences, observed differences in incidence and disease aggressiveness at presentation indicate a potential role for different pathways of prostate carcinogenesis between men of AD and CD.

Over the past decade, much research has focused on mutations of cancer genes and their effects, including the identification of germline mutations having clinical utility that can be used in the prediction, management and treatment of cancers. For example, the Myriad myRisk® Hereditary Cancer, Invitae® Cancer Screen, Centogene’s CentoCancer® Comprehensive Cancer Panel, and Ambry Genetic’s CancerNextX® are all commercially-available products that use next-generation sequencing to predict the risk of cancer development, based on analysis of specific genes thought to be related to inherited cancers, including, for example, prostate cancer. Nonetheless, a need exists to identify novel gene panels that can be used to predict a future cancer occurrence, diagnose cancer, or prognose cancer across a variety of ethnicities. Therefore, new biomarkers and therapeutic markers that are specific for distinct ethnic populations (e.g., African or Caucasian descent) and provide more accurate diagnostic and/or prognostic potential are needed.

### SUMMARY

The present disclosure provides gene panels that are associated with prostate cancer and methods of using the same. The gene panels can be used to predict an elevated risk of developing prostate cancer. In one aspect, the gene panel is specific for patients of African descent, and in one aspect, the gene panel is specific for patients of Caucasian descent. In one aspect, the gene panel is specific for patients having a family history of cancer, such as a family history of prostate cancer or breast cancer. In another aspect, the gene panel provides similar sensitivity/specificity of cancer prediction and/or detection in patients of both African and Caucasian descent.

In one aspect is disclosed a method of predicting a predisposition for developing prostate cancer in a patient, the method comprising assaying a biological sample obtained from the patient to determine if the biological sample contains at least one pathogenic or likely pathogenic gene mutation in a plurality of genes, wherein the plurality of genes comprises the following human genes: BRCA1, PMS2, RAD51, RAD54B, and RAD54L, wherein the patient is identified as having a predisposition for developing prostate cancer if a pathogenic or likely pathogenic gene mutation is detected in at least one of BRCA1, PMS2, RAD51, RAD54B, or RAD54L.

In another aspect, there is disclosed a method of obtaining a gene mutation profile in a biological sample from a patient, the method comprising assaying a biological sample obtained from the patient to determine if the biological sample contains at least one pathogenic or likely pathogenic gene mutation in a plurality of genes, wherein the plurality of genes comprises the following human genes BRCA1, PMS2, RAD51, RAD54B, and RAD54L.

In certain embodiments of the methods herein, the plurality of genes further comprises at least 10, such as at least 15, at least 20, at least 25, at least 35, at least 40, or all of the following 42 human genes: ATM, ATR, BLM, BRCA2, CHEK2, DNA2, ERCC2, ERCC3, ERCC4, ERCC6, FAN1, FANCA, FANCC, FANCD2, FANCI, FANCL, GTF2H5, HFM1, IDH1, IN080, LIG1, MLH3, MSH2, MSH6, MUTYH, NBN, NTHL1, OGG1, PCNA, PNKP, POLG, POLH, POLK, RAD51C, RRM2B, TDP2, TP53, TELO2, TTK, TUBGCP4, UNG, and XPA. In certain embodiments, the plurality of genes further comprises at least 8, such as at least 10, such as at least 15, or all 20 of the following 20 human genes: ATM, BRCA2, CHEK2, ERCC2, FAN1, FANCA, FANCC, FANCD2, FANCI, FANCL, GTF2H5, MLH3, MSH2, MSH6, MUTYH, NBN, OGG1, POLG, POLH, and RAD51C. In certain embodiments, the plurality of genes further comprises at least 5, such as at least 10, or all 14 of the following 14 human genes: ATM, CHEK2, ERCC2, FAN1, FANCA, FANCD2, FANCL, GTF2H5, MSH6, MUTYH, NBN, OGG1, POLG, and POLH. In certain embodiments, the plurality of genes further comprises at least 4, such as 5, such as at least 10, or all 11 of the following 11 human genes: BRCA2, FAN1, FANCA, FANCC, FANCD2, FANCI, FANCL, MLH, MSH2, MSH6, and RAD51C. In certain embodiments, the plurality of genes further comprises at least one, at least 3, at least 5, or all 8 of the following 8 human genes: CHEK2, ERCC2, FANCA, FANCL, MSH6, MUTYH, OGG1, and POLG. In certain embodiments, the plurality of genes further comprises at least one, such as 2, or all 3 of the following 3 human genes: FANCA, FANCL, and MSH6.

In another aspect, there is a method for selecting a treatment for a patient with prostate cancer, the method comprising:
assaying a biological sample from the patient to determine if the biological sample contains at least one pathogenic or likely pathogenic gene mutation from a plurality of human genes, wherein the plurality of human genes comprises BRCA1, PMS2, RAD51, RAD54B, and RAD54L (“the 5-gene panel,” as defined herein); and
selecting a treatment for the patient if at least one pathogenic or likely pathogenic gene mutation is detected in the plurality of human genes, wherein the selected treatment comprises surgery, radiation, hormone therapy, chemotherapy, biological therapy, or high intensity focused ultrasound. In another embodiment, the plurality of human genes comprises: at least 15, such as at least 20, at least 25, at least 30, at least 35, at least 40, at least 45, or all 47 genes of the 47-gene panel (as defined herein); at least 13, such as at least 15, at least 20, or all 25 genes of the 25-gene panel (as defined herein); at least 10, such as at least 15, or all 19 genes of the 19-gene panel (as defined herein); at least 9, such as at least 10, at least 15, or all 16 genes of the 16-gene panel (as defined herein); at least 8, such as at least 10, or all 13 genes of the 13-gene panel (as defined herein); or at least 6, such as at least 7, or all 8 genes of the 8-gene panel (as defined herein).

In some aspects, the selected treatment can comprise a therapy that induces DNA damage and/or apoptosis, such as, radiation, a poly(ADP ribose) polymerase (PARP) inhibitor, or a platinum-based therapeutic.

In some aspects, the selected treatment may comprise target therapies, wherein the targeted therapies comprise using one or more therapeutics that specifically target the pathogenic or likely pathogenic DDRGs identified in a subject suffering from prostate cancer.

In another aspect, there is a method for stratifying prostate cancer in a patient, the method comprising:
assaying a biological sample from the patient to determine if the biological sample contains at least one pathogenic or likely pathogenic gene mutation from a plurality of human genes, wherein the plurality of human genes comprises BRCA1, PMS2, RAD51, RAD54B, and RAD54L (“the 5-gene panel,” as defined herein); and
stratifying the prostate cancer patients into distinct molecular subtypes with different prognosis and identifying those having an increased risk of biochemical recurrence following radical prostatectomy if at least one pathogenic or likely pathogenic gene mutation is detected in the plurality of human genes. In certain embodiments, the plurality of human genes comprises: at least 15, such as at least 20, at least 25, at least 30, at least 35, at least 40, at least 45, or all 47 genes of the 47-gene panel (as defined herein); at least 13, such as at least 15, at least 20, or all 25 genes of the 25-gene panel (as defined herein); at least 10, such as at least 15, or all 19 genes of the 19-gene panel (as defined herein); at least 9, such as at least 10, at least 15, or all 16 genes of the 16-gene panel (as defined herein); at least 8, such as at least 10, or all 13 genes of the 13-gene panel (as defined herein); or at least 6, such as at least 7, or all 8 genes of the 8-gene panel (as defined herein).

In various aspects of the methods disclosed herein, including, for example, in methods involving the 16-gene panel, the 8-gene panel, and the 5-gene panel, the patient is of African descent, and in certain aspects, the patient has a family history of cancer, such as prostate cancer or breast cancer or ovarian cancer.

In certain embodiments of all aspects of the present disclosure, the biological sample is assayed using sequencing techniques, and in certain embodiments, each of the genes in the plurality of genes is sequenced before determining if the biological sample contains at least one pathogenic or likely pathogenic gene mutation in a plurality of genes.

In certain embodiments of all aspects of the present disclosure, the assaying step comprises detecting nucleic acid expression and in certain embodiments, the assaying step comprises detecting polypeptide expression.

In certain embodiments of all aspects of the present disclosure, the biological sample comprises the patient’s blood or saliva or urine or other body fluid or is obtained therefrom.

In certain embodiments of all aspects of the present disclosure, the methods further comprise a step of providing genetic counseling to the patient. In certain embodiments, the patient has a family history of cancer, such as a family history of DDRG germline mutation related cancer, including prostate cancer or breast cancer.

In certain embodiments of all aspects of the present disclosure, if at least one pathogenic or likely pathogenic gene mutation is detected in the plurality of genes, the method further comprises a step of treating the patient. In certain embodiments, the treatment comprises surgery, radiation, hormone therapy, chemotherapy, biological therapy, targeted therapy, or high intensity focused ultrasound. In certain embodiments, the treatment is a therapy that induces DNA damage and/or apoptosis, such as radiation, a poly(ADP ribose) polymerase (PARP) inhibitor, or a platinum-based therapeutic. The treatment may also comprise targeted therapies, wherein the targeted therapies comprise using one or more therapeutics that specifically target the pathogenic or likely pathogenic DDRGs identified in a subject suffering from prostate cancer.

Another aspect is directed to kits for use in predicting, diagnosing, and/or prognosing cancer, the kit comprising a plurality of probes for detecting a pathogenic or likely pathogenic gene mutation in the following human genes: BRCA1, PMS2, RAD51, RAD54B, and RAD54L, wherein the plurality of probes contains probes for detecting the pathogenic or likely pathogenic gene mutation in no more than 500 different genes. In certain embodiments, the plurality of probes further comprises probes for at least 10, such as at least 15, at least 20, at least 25, at least 30, at least 35, at least 40, or all 42 of the following 42 human genes: ATM, ATR, BLM, BRCA2, CHEK2, DNA2, ERCC2, ERCC3, ERCC4, ERCC6, FAN1, FANCA, FANCC, FANCD2, FANCI, FANCL, GTF2H5, HFM1, IDH1, INO80, LIG1, MLH3, MSH2, MSH6, MUTYH, NBN, NTHL1, OGG1, PCNA, PNKP, POLG, POLH, POLK, RAD51C, RRM2B, TDP2, TP53, TELO2, TTK, TUBGCP4, UNG, and XPA.

In another aspect, plurality of probes in the kit for use in predicting, diagnosing, and/or prognosing cancer further comprises probes for detecting a pathogenic or likely pathogenic mutation in at least 8, such as at least 10, at least 15, or all 20 of the following 20 human genes: ATM, BRCA2, CHEK2, ERCC2, FAN1, FANCA, FANCC, FANCD2, FANCI, FANCL, GTF2H5, MLH3, MSH2, MSH6, MUTYH, NBN, OGG1, POLG, POLH, and RAD51C.

In certain embodiments, the plurality of probes in the kit for use in predicting, diagnosing, and/or prognosing cancer further comprises probes for detecting a pathogenic or likely pathogenic mutation in at least 5, such as at least 10, or all 14 of the following 14 human genes: ATM, CHEK2, ERCC2, FAN1, FANCA, FANCD2, FANCL, GTF2H5, MSH6, MUTYH, NBN, OGG1, POLG, and POLH.

In certain embodiments, the plurality of probes in the kit for use in predicting, diagnosing, and/or prognosing cancer further comprises probes for detecting a pathogenic or likely pathogenic mutation in at least 4, such as at least 5, at least 10, or all 11 of the following 11 human genes: BRCA2, FAN1, FANCA, FANCC, FANCD2, FANCI, FANCL, MLH, MSH2, MSH6, and RAD51C.

In certain embodiments, the plurality of probes in the kit for use in predicting, diagnosing, and/or prognosing cancer further comprises probes for detecting a pathogenic or likely pathogenic mutation in at least 3, such as at least 5, or all 8 of the following 8 human genes: CHEK2, ERCC2, FANCA, FANCL, MSH6, MUTYH, OGG1, and POLG.

In certain embodiments, the plurality of probes in the kit for use in predicting, diagnosing, and/or prognosing cancer further comprises probes for detecting a pathogenic or likely pathogenic mutation in at least one, such as at least 2, or all 3 of the following 3 human genes: FANCA, FANCL, and MSH6.

In some embodiments of all aspects of the present disclosure, the plurality of probes is selected from a plurality of oligonucleotide probes, a plurality of antibodies, or a plurality of polypeptide probes. In some embodiments of all aspects of the present disclosure, the plurality of probes contains probes for detecting pathogenic or likely pathogenic gene mutations in no more than 250, 100, 75, 60, 50, 47, 40, 30, 25, 20, 19, 16, 15, 13, 9, 10, 8, 6, or 5 different genes.

In some embodiments of all aspects of the present disclosure, the plurality of probes is attached to the surface of an array, and in yet another aspect, the array comprises no more than 250, 100, 75, 60, 50, 47, 40, 30, 25, 20, 19, 16, 15, 13, 9, 10, 8, 6, or 5 different addressable elements. In some embodiments of all aspects of the present disclosure, the plurality of probes is labeled.

In some embodiments of all aspects of the present disclosure, there is a genetic testing method for identifying a patient having a predisposition for developing prostate cancer, the method comprising obtaining a biological sample from the patient and assaying the biological sample to determine if the biological sample contains at least one pathogenic or likely pathogenic gene mutation from a plurality of genes, wherein the plurality of genes comprises the following human genes: BRCA1, PMS2, RAD51, RAD54B, and RAD54L, wherein the patient is identified as having a predisposition for developing prostate cancer if at least one pathogenic or likely pathogenic mutation is detected in at least one of BRCA1, PMS2, RAD51, RAD54B, or RAD54L. In certain embodiments, prior to assaying the biological sample, the patient is identified as having a family history of cancer, such as a family history of DDRG germline mutation related cancer, including prostate cancer or breast cancer. In certain embodiments, the patient is of African descent.

In some embodiments of all aspects of the present disclosure, there is a method of characterizing prostate cancer in a patient comprising assaying a biological sample obtained from the patient to determine if the biological sample contains at least one pathogenic or likely pathogenic gene mutation in a plurality of genes, wherein the plurality of genes comprises the following human genes: BRCA1, PMS2, RAD51, RAD54B, and RAD54L, wherein detecting the presence of at least one pathogenic or likely pathogenic gene mutation in at least one of BRCA1, PMS2, RAD51, RAD54B, or RAD54L characterizes the prostate cancer in the subject as being an aggressive form of prostate cancer or as having an increased risk of developing into an aggressive form of prostate cancer. In certain embodiments, the patient is of African descent.

### BRIEF DESCRIPTION OF THE DRAWINGS

The accompanying drawings, which are included to provide a further understanding of the disclosure, are incorporated in and constitute a part of this specification. The drawings illustrate embodiments of the disclosure and, together with the detailed description, serve to explain the principles of the disclosure. No attempt is made to show structural details of the disclosure in more detail than may be necessary for a fundamental understanding of the disclosure and various ways in which it may be practiced.

FIG. 1 is a bar graph illustrating the pathogenic variant carrier rate for 47 DDRGs in prostate cancer samples from both African-American (left) and Caucasian-American (right) patients.

FIG. 2 is a Kaplan-Meier plot showing the survival probability of African-American patients exhibiting a biochemical recurrence of prostate cancer over time as a function of DDGR germline mutation, wherein African-American patients having at least one DDRG germline mutation are shown to have a lower survival probability over 20 months than African-American patients who did not have any DDRG germline mutations.

FIG. 3A is a graph showing the correlation between the percentage of allele frequency in the Center for Prostate Disease Research (CPDR) database as compared to the percentage of allele frequency in the public Exome Aggregation Consortium (ExAC) database for African-American men, as discussed in Example 1.

FIG. 3B is a graph showing the correlation between the percentage of allele frequency in the CPDR database as compared to the percentage of allele frequency in the public ExAC database for Caucasian men, as discussed in Example 1.

FIG. 4 is a schematic diagram illustrating MDC1, a co-activator of Androgen Receptor (AR), acting as an AR-induced transactivator and suppressor of prostate cancer.

FIG. 5 is a volcano plot showing a single non-silent variant association test of CPDR African-American men versus ExAC African-American men, wherein each dot on the plot represents a single non-silent variant and labeled red dots represent variants having a false discovery rate (FDR) < 0.05.

The drawings are not necessarily to scale, and may, in part, include exaggerated dimensions for clarity.

### DETAILED DESCRIPTION

Reference will now be made in detail to various exemplary embodiments, examples of which are illustrated in the accompanying drawings. It is to be understood that the following detailed description is provided to give the reader a fuller understanding of certain embodiments, features, and details of aspects of the invention, and should not be interpreted as a limitation of the scope of the invention.

Disclosed herein are methods for predicting, diagnosing and prognosing cancer, such as prostate cancer. Mutation of DDRGs are known to be involved in prostate cancer development. Nicolosi, P. et al., Prevalence of Germline Variants in Prostate Cancer and Implications for Current Genetic Testing Guidelines, J. Am. Med. Assoc. Oncol. 2019; doi:10.1001/jamaoncol.2018.6760. Cellular DNA is continually under attack and subject to damage by various environment and intracellular agents. This DNA damage may be minimized or reversed, however, by the actions of various known DDRGs, which may serve, for example, to repair double and single stranded DNA breaks through mechanisms including base excision repair, nucleotide excision repair, mismatch repair, and homologous recombination. Wood, R.D. et al., Human DNA Repair Genes, SCIENCE 2001; 291:1284-1289.

As the existence of germline mutations affecting DDRGs may significantly increase a patient’s likelihood of developing cancer or affect a cancer patient’s prognosis, identifying DDRG germline mutations involved in cancer, such as prostate cancer, may be a useful tool in predicting the development, diagnosis, and prognosis of cancer. Moreover, DDRGs involved in the homologous recombination pathway or the mismatch repair pathway may be of particular interest, as these DDRGs are considered targetable by known therapeutics, e.g., by poly(ADP ribose) polymerase (PARP) inhibitors for DDRGs involved in the homologous recombination pathway and by immune checkpoint inhibitors for DDRGs involved in the mismatch repair pathway. Accordingly, disclosed herein are gene panels that may be used in methods to obtain specific pathogenic or likely pathogenic gene mutation profiles and to use those gene mutation profiles to predict, diagnose, and prognose prostate cancer, particularly in patients having a history of cancer or across patients of different ethnicities.

### Definitions

In order that the present invention may be more readily understood, certain terms are first defined. Additional definitions are set forth throughout the detailed description.

The term “of African descent” refers to individuals who self-identify as being of African descent, including individuals who self-identify as being African-American, and individuals determined to have genetic markers correlated with African ancestry, also called Ancestry Informative Markers (AIM), such as the AIMs identified in Judith Kidd et al., Analyses of a set of 128 ancestry informative single-nucleotide polymorphisms in a global set of 119 population samples, INVESTIGATIVE GENETICS, (2):1, 2011, which reference is incorporated by reference in its entirety.

The term “of Caucasian descent” refers to individuals who self-identify as being of Caucasian descent, including individuals who self-identify as being Caucasian-American, and individuals determined to have genetic markers correlated with Caucasian ancestry, such as European, North African, or Asian (Western, Central or Southern) ancestry, also called Ancestry Informative Markers (AIM), such as the AIMs identified in Judith Kidd et al., Analyses of a set of 128 ancestry informative single-nucleotide polymorphisms in a global set of 119 population samples, INVESTIGATIVE GENETICS, (2):1, 2011, which reference is incorporated by reference in its entirety.

The term “antibody” refers to an immunoglobulin or antigen-binding fragment thereof, and encompasses any polypeptide comprising an antigen-binding fragment or an antigen-binding domain. The term includes but is not limited to polyclonal, monoclonal, monospecific, polyspecific, humanized, human, single-chain, chimeric, synthetic, recombinant, hybrid, mutated, grafted, and in vitro generated antibodies. Unless preceded by the word “intact,” the term “antibody” includes antibody fragments such as Fab, F(ab′)₂, Fv, scFv, Fd, dAb, and other antibody fragments that retain antigen-binding function. Unless otherwise specified, an antibody is not necessarily from any particular source, nor is it produced by any particular method.

The term “detecting” or “detection” means any of a variety of methods known in the art for determining the presence or amount of a nucleic acid or a protein. As used throughout the specification, the term “detecting” or “detection” includes either qualitative or quantitative detection.

The term “isolated,” when used in the context of a polypeptide or nucleic acid refers to a polypeptide or nucleic acid that is substantially free of its natural environment and is thus distinguishable from a polypeptide or nucleic acid that might happen to occur naturally. For instance, an isolated polypeptide or nucleic acid is substantially free of cellular material or other polypeptides or nucleic acids from the cell or tissue source from which it was derived.

The term “therapeutically effective amount” refers to a dosage or amount that is sufficient for treating an indicated disease or condition.

The terms “polypeptide,” “peptide,” and “protein” are used interchangeably herein to refer to polymers of amino acids.

The term “polypeptide probe” as used herein refers to a labeled (e.g., isotopically labeled) polypeptide that can be used in a protein detection assay (e.g., mass spectrometry) to quantify a polypeptide of interest in a biological sample.

The term “primer” means a polynucleotide capable of binding to a region of a target nucleic acid, or its complement, and promoting nucleic acid amplification of the target nucleic acid. Generally, a primer will have a free 3′ end that can be extended by a nucleic acid polymerase. Primers also generally include a base sequence capable of hybridizing via complementary base interactions either directly with at least one strand of the target nucleic acid or with a strand that is complementary to the target sequence. A primer may comprise target-specific sequences and optionally other sequences that are non-complementary to the target sequence. These non-complementary sequences may comprise, for example, a promoter sequence or a restriction endonuclease recognition site.

A “mutation” or “mutant” refers to an allele sequence that is different from the reference at as little as a single base or for a longer interval. Mutants, also referred to herein as variants, may be classified as pathogenic, likely pathogenic, uncertain significance, likely benign, or benign, as classified in the Joint Consensus Recommendation of the American College of Medical Genetics and Genomics and the Association for Molecular Pathology Standards and Guidelines for the Interpretation of Sequence Variants. Richards, S. et al., Standards and Guidelines for the Interpretation of Sequence Variants: A Joint Consensus Recommendation of the American College of Medical Genetics and Genomics and the Association for Molecular Pathology, GENET. MED. 2015; 17(5):405-424; see also Chirita-Emandi, A. et al., Challenges in reporting pathogenic/potentially pathogenic variants in 94 cancer predisposing genes - in pediatric patients screened with NGS panels, Scientific Reports 2020; 10(1):1-9. A pathogenic (P) gene mutation indicates that the variant contributes to the development of the disease, while a likely pathogenic (LP) gene mutation indicates that there is a high probability (e.g., greater than 90% certainty) that the variant contributes to the development of the disease. See Richards 2015, discussing an established five-tiered guidance for categorizing variants as pathogenic, likely pathogenic, uncertain significance, likely benign, and benign. Pathogenic/likely pathogenic (P/LP) gene mutations refer to variants that are either pathogenic or likely pathogenic.

The term “gene mutation profile” refers to the presence or absence of mutations in a plurality of genes in a sample as compared to the wild-type genes. As is understood in the art, mutations of a gene can be analyzed through sequencing techniques or by measuring the expression of a nucleic acid (e.g., genomic DNA or mRNA) or a polypeptide that is encoded by the nucleic acid.

The term “gene panel” refers to one or more genes or groups of genes wherein the presence of a pathogenic or likely pathogenic mutation in any one of the genes of the gene panel may indicate a predisposition towards the development of a pathological condition, such as cancer.

The term “5-gene panel” refers to the following 5 human genes: BRCA1, PMS2, RAD51, RAD54B, and RAD54L.

The term “47-gene panel” refers to the following 47 human genes: ATM, ATR, BLM, BRCA1, BRCA2, CHEK2, DNA2, ERCC2, ERCC3, ERCC4, ERCC6, FAN1, FANCA, FANCC, FANCD2, FANCI, FANCL, GTF2H5, HFM1, IDH1JNO80, LIG1, MLH3, MSH2, MSH6, MUTYH, NBN, NTHL1, OGG1, PCNA, PMS2, PNKP, POLG, POLH, POLK, RAD51, RAD51C, RAD54B, RAD54L, RRM2B, TDP2, TP53, TELO2, TTK, TUBGCP4, UNG, and XPA.

The term “25-gene panel” refers to the following 25 human genes: ATM, BRCA1, BRCA2, CHEK2, ERCC2, FAN1, FANCA, FANCC, FANCD2, FANCI, FANCL, GTF2H5, MLH3, MSH2, MSH6, MUTYH, NBN, OGG1, PMS2, POLG, POLH, RAD51, RAD54B, RAD54L, and RAD51C.

The term “19-gene panel” refers to the following 19 human genes: ATM, BRCA1, CHEK2, ERCC2, FAN1, FANCA, FANCD2, FANCL, GTF2H5, MSH6, MUTYH, NBN, OGG1, PMS2, POLG, POLH, RAD51, RAD54B, and RAD54L.

The term “16-gene panel” refers to the following 16 human genes: BRCA1, BRCA2, FAN1, FANCA, FANCC, FANCD2, FANCI, FANCL, MLH, MSH2, MSH6, PMS2, RAD51, RAD51C, RAD54B, and RAD54L.

The term “13-gene panel” refers to the following 13 human genes: BRCA1, CHEK2, ERCC2, FANCA, FANCL, MSH6, MUTYH, OGG1, PMS2, POLG, RAD51, RAD54B, and RAD54L.

The term “8-gene panel” refers to the following 8 human genes: BRCA1, FANCA, FANCL, MSH6, PMS2, RAD51, RAD54B, and RAD54L.

The term “carrier frequency” refers to a percentage of patient samples having a single copy of a specific recessive gene mutation in a pool of patient samples. A germline mutation having a carrier frequency greater than a given percentage, such as greater than 0.5% or greater than 1%, may indicate a germline mutation that can be used to predict, diagnose, or prognose cancer, such as prostate cancer.

As used herein, a “biochemical recurrence” (BCR) refers to a post-radical prostatectomy serum prostate-specific antigen (PSA) increase that indicates treatment by hormonal ablation and/or chemotherapy. The PSA increase is typically a PSA greater than or equal to 0.1 ng/mL, or a PSA greater than or equal to 0.2 ng/mL, measured no less than eight weeks after radical prostatectomy, followed by a successive, confirmatory PSA level greater than or equal to 0.2 ng/mL.

Where available, HUGO Gene Nomenclature Committee (HGNC) annotations are used to describe the genes discussed herein, as well as Ensembl gene annotations. The following Table 1 lists the HGNC annotations, Ensemble gene annotations, Entrezgene numbers, and/or gene name descriptions for the genes discussed herein, where available:

TABLE 1

HGNC and Ensembl Gene Annotations

| HGNC Symbol | HGN C ID | Ensembl Annotation | Entre z gene No. | NCBI Reference | Description |
|---|---|---|---|---|---|
| ATM | 795 | ENSG0000 0149311.18 | 472 | NM_000051.3 GI: 71902539 | ATM serine/threonine kinase |
| ATR | 882 | ENSG0000 0175054.16 | 545 | NM_001184.4 GI: 1519245580 | ATR serine/threonine kinase |
| BLM | 1058 | ENSG0000 0197299.12 | 641 | NM_000057.4 GI: 1519245724 | BLM RecQ like helicase |
| BRCA1 | 1100 | ENSG0000 0012048.22 | 672 | NM_007294.4 GI: 1732746264 | BRCA1 DNA repair associated |
| BRCA2 | 1101 | ENSG0000 0139618.15 | 675 | NM_000059.3 | BRCA2 DNA repair associated |
| CHEK2 | 16627 | ENSG0000 0183765.22 | 11200 | NM_001005735.2 GI: 1675053443 | Checkpoint kinase 2 |
| DNA2 | 2939 | ENSG0000 138346.10 | 1763 | NM_001080449.3 GI: 1519244522 | DNA replication helicase/nuclease 2 |
| ERCC2 | 3434 | ENSG0000 0104884.15 | 2068 | NM_000400.4 GI: 1777425446 | ERCC excision repair 2, TFIIH core complex helicase subunit |
| ERCC3 | 3435 | ENSG0000 0163161.13 | 2071 | NM_000122.2 GI: 1732746215 | ERCC excision repair 3, TFIIH core complex helicase subunit |
| ERCC4 | 3436 | ENSG0000 0175595.14 | 2072 | NM_005236.3 GI: 173274629 | ERCC excision repair 4, endonuclease catalytic subunit |
| ERCC6 | 3438 | ENSG0000 0225830.13 | 2074 | NM_000124.4 GI: 1519246499 | ERCC excision repair 6, chromatin remodeling factor |
| FAN1 | 29170 | ENSG0000 0198690.10 | 22909 | NM_014967.5 GI: 1653962357 | FANCD2 and FANCI associated nuclease 1 |
| FANCA | 3582 | ENSG0000 0187741.15 | 2175 | NM_000135.4 GI: 1519243346 | FA complementation group A |
| FANCC | 3584 | ENSG0000 0158169.13 | 2176 | NM_000136.3 GI: 1653962344 | FA complementation group C |
| FANCD 2 | 3585 | ENSG0000 0144554.10 | 2177 | NM_001018115.3 GI: 1741710897 | FA complementation group D2 |
| FANCI | 25568 | ENSG0000 0140525.17 | 55215 | NM_018193.3 GI: 1677538557 | FA complementation group I |
| FANCL | 20748 | ENSG0000 0115392.11 | 55120 | NM_018062.4 GI: 1776926126 | FA complementation group L |
| GTF2H 5 | 21157 | ENSG0000 0272047.2 | 40467 2 | NM_207118.3 GI: 1732746154 | general transcription factor IIH subunit 5 |
| HFM1 | 20193 | ENSG0000 0162669.11 | 16404 5 | NM_001017975.6 GI: 1804891963 | helicase for meiosis 1, ATP dependent DNA helicase homolog |
| IDH1 | 5382 | ENSG0000 0138413.13 | 3417 | NM_005896.4 GI: 538917457 | Isocitrate dehydrogenase (NADP(+)) 1 |
| INO80 | 26956 | ENSG0000 0128908.17 | 54617 | NM_017553.3 GI: 1394370137 | INO80 complex ATPase subunit |
| LIG1 | 6598 | ENSG0000 0105486.14 | 3978 | NM_000234.3 GI: 1519312512 | DNA ligase 1 |
| MDC1 | 21163 | ENSG0000 0137337.14 | 9656 | NM_014641.3 GI: 1519242572 | mediator of DNA damage checkpoint 1 |
| MLH3 | 7128 | ENSG0000 0119684.15 | 27030 | NM_014381.3 GI: 1677530627 | mutL homolog 3 |
| MSH2 | 7325 | ENSG0000 0095002.14 | 4436 | NM_000251.3 GI: 1732746316 | mutS homolog 2 |
| MSH6 | 7329 | ENSG0000 0116062.15 | 2956 | NM_000179.3 GI: 1778901296 | mutS homolog 6 |
| MUTY H | 7527 | ENSG0000 0132781.18 | 4595 | NM_012222.2 GI: 115298646 | mutY DNA glycosylase |
| NBN | 7652 | ENSG0000 0104320.13 | 4683 | NM_001024688.2 GI: 800924417 | Nibrin |
| NTHL1 | 8028 | ENSG0000 0065057.9 | 4913 | NM_002528.7 GI: 1514769206 | nth like DNA glycosylase 1 |
| OGG1 | 8125 | ENSG0000 0114026.21 | 4968 | NM_016821.2 GI: 197276617 | 8-oxoguanine DNA glycosylase |
| PCNA | 8729 | ENSG0000 0132646.11 | 5111 | NM_002592.2 GI: 33239449 | proliferating cell nuclear antigen |
| PMS2 | 9122 | ENSG0000 0122512.16 | 5395 | NM_000535.7 GI: 1519311653 | PMS1 homolog 2, mismatch repair system component |
| PNKP | 9154 | ENSG0000 0039650.12 | 11284 | NM_007254.4 | polynucleotide kinase 3′-phosphatase |
| POLG | 9179 | ENSG0000 0140521.7 | 5428 | NM_002693.2 GI: 1804891999 | DNA polymerase gamma |
| POLH | 9181 | ENSG0000 0170734.11 | 5429 | NM_006502.3 GI: 1732746153 | DNA polymerase eta |
| POLK | 9183 | ENSG0000 0122008.15 | 51426 | NM_016218.3 GI: 1066559737 | DNA Polymerase kappa |
| RAD51 | 9817 | ENSG0000 0051180.17 | 5888 | NM_002875.5 GI: 1653960901 | RAD51 recombinase |
| RAD51 C | 9820 | ENSG0000 0108384.15 | 5889 | NM_058216.3 GI: 1519243717 | RAD51 paralog C |
| RAD54 B | 17228 | ENSG0000 0197275.14 | 25788 | NM_012415.3 GI: 327532736 | RAD54B homolog |
| RAD54 L | 9826 | ENSG0000 0085999.13 | 8438 | NM_003579.4 GI: 1519244518 | RAD54 like |
| RRM2B | 17296 | ENSG0000 0048392.11 | 50484 | NM_001172477.1 GI: 289177073 | ribonucleotide reductase regulatory TP53 inducible subunit M2B |
| TDP2 | 17768 | ENSG0000 0111802.14 | 51567 | NM_016614.3 | tyrosyl-DNA phosphodiesterase 2 |
| TELO2 | 29099 | ENSG0000 0100726.15 | 9894 | NM_016111.4 GI: 1519313627 | telomere maintenance 2 |
| TP53 | 11998 | ENSG0000 0141510.17 | 7157 | NM_000546.5 GI: 371502114 | Tumor protein p53 |
| TTK | 12401 | ENGS0000 0112742.10 | 7272 | NM_003318.5 GI: 1519313962 | TTK protein kinase |
| TUBGC P4 | 16691 | ENGS0000 0137822.12 | 27229 | NM_014444.5 GI: 1677538566 | tubulin gamma complex associated protein 4 |
| UNG | 12572 | ENSG0000 0076248.10 | 7374 | NM_080911.3 GI: 1677498868 | uracil DNA glycosylase |
| XPA | 12814 | ENSG0000 0136936.10 | 7507 | NM_000380.4 GI: 1779521776 | XPA, DNA damage recognition and repair factor |

The terms “prognosis” and “prognosing” as used herein mean predicting the likelihood of death from the cancer and/or recurrence or metastasis of the cancer within a given time period or predicting the likelihood of developing cancer during the patient’s lifetime, with or without consideration of the likelihood that the cancer patient will respond favorably or unfavorably to a chosen therapy or therapies.

As used herein, “genetic testing” refers to a type of medical test that identifies changes in chromosomes, genes, or proteins. The results of a genetic test can confirm or rule out a suspected genetic condition or help determine a person’s chance of developing or passing on a genetic disorder. “Genetic testing” also refers to the process of analyzing cells or tissue to look for changes in genes, chromosomes, or proteins that may be a sign of a disease or condition, such as cancer. These changes may also be a sign that a person has an increased risk of developing a specific disease or condition. Genetic testing may be done on tumor tissue to help diagnose cancer, plan treatment, or find out how well treatment is working.

As used herein, “genetic counseling” refers to a communication process between a specially trained health professional and a person concerned about the genetic risk of disease. The person’s family and personal medical history may be discussed, and counseling may lead to genetic testing or pertain to the results of genetic testing.

In the specification, the term “biological sample” should be understood to mean any sample obtained from a patient where germline mutations can be detected, including tumor cells and non-tumor cells, such as prostate cells, blood or blood derivatives (serum, plasma, etc.), saliva, semen or seminal fluid, urine, or cerebrospinal fluid.

As used herein, the term “fragment” means a part or portion of a polynucleotide sequence comprising about 10 or more contiguous nucleotides, about 15 or more contiguous nucleotides, about 20 or more contiguous nucleotides, about 30 or more, or even about 50 or more contiguous nucleotides. In certain embodiments, the polynucleotide probes will comprise 10 or more nucleic acids, 20 or more, 50 or more, or 100 or more nucleic acids. In order to confer sufficient specificity, the probe may have a sequence identity to a complement of the target sequence of about 90% or more, such as about 95% or more (e.g., about 98% or more or about 99% or more) as determined, for example, using the well-known Basic Local Alignment Search Tool (BLAST) algorithm (available through the National Center for Biotechnology Information (NCBI), Bethesda, Md.).

### Detecting Germline Mutations

Assaying a sample to detect a germline mutation in a gene, such as any of the genes in the gene panels disclosed herein, comprises measuring or detecting any nucleic acid transcript (e.g., mRNA, cDNA, or genomic DNA) that evidences the germline mutation or any protein encoded by such a nucleic acid transcript, if applicable. The presence or absence of the germline mutation can be measured or detected by measuring or detecting one or more of the genomic sequences or mRNA/cDNA transcripts corresponding to the target gene mutation, or to all of the genomic sequences or mRNA/cDNA transcripts associated with the target gene.

Germline mutations can be detected by any method known in the art, including but not limited to DNA-sequencing (DNA-seq), RNA-sequencing (RNA-seq), polymerase chain reaction (PCR), fluorescent in situ hybridization (FISH) analysis, and chromogenic in situ hybridization (CISH). For example, FISH analysis can be used to detect chromosomal rearrangements. In these embodiments, nucleic acid probes that hybridize under conditions of high stringency to the chromosomal mutation are incubated with a biological sample comprising somatic cells (or nucleic acid obtained therefrom). Other known in situ hybridization techniques can be used to detect mutations. The nucleic acid probes (DNA or RNA) can hybridize to DNA or mRNA and can be designed to detect germline mutations, including deletions such as single base pair deletions, insertions, duplications, fusions, inversions, and amino acid changes.

Typically, the nucleic acid probes are labeled to assist with detection of hybridization to a target sequence. Such labeled nucleic acid probes do not occur naturally. As used herein, DNA-seq refers to any high-throughput sequencing technique used to detect the presence and quantity of DNA in a sample. DNA-seq can be used to identify genomic variants and rearrangements, including, for example, deletions, insertions, duplications, fusions, and inversions. For example, in some embodiments, high-throughput sequencing techniques may be used to sequence relatively short fragments of sample DNA, which may then be mapped to a reference genome to identify gene mutations.

As used herein, detecting a mutation or the expression of any of the foregoing genes or nucleic acids may comprise measuring or detecting any nucleic acid transcript (e.g., mRNA, cDNA, or genomic DNA) corresponding to the gene mutation of interest or the protein encoded thereby. The presence or absence of a gene mutation may be detected by measuring or detecting the expression of a gene mutation or nucleic acids corresponding to the same, for example if the gene mutation or nucleic acids corresponding to the same are not detected, or if the measurement of the expression of the gene mutation or nucleic acids corresponding to the same falls below a threshold level, the gene mutation or nucleic acids corresponding to the same may be determined to be absent. Likewise, if the gene mutation or nucleic acids corresponding to the same are detected, or if the measurement of the expression of the gene mutation or nucleic acids corresponding to the same falls above a threshold level, the gene mutation or nucleic acids corresponding to the same may be determined to be present. If a gene mutation is associated with more than one mRNA transcript or isoform, the expression of the gene mutation can be measured or detected by measuring or detecting one or more of the mRNA transcripts, or all of the mRNA transcripts associated with the gene mutation.

Typically, gene expression can be detected or measured on the basis of mRNA or cDNA levels, although protein levels also can be used when appropriate. Any quantitative or qualitative method for measuring mRNA levels, cDNA, or protein levels can be used. Suitable methods of detecting or measuring mRNA or cDNA levels include, for example, Northern Blotting, RNAse protection assays, microarray analysis, RNA-sequencing, or a nucleic acid amplification procedure, such as reverse-transcription PCR (RT-PCR) or real-time RT-PCR, also known as quantitative RT-PCR (qRT-PCR). Such methods are well known in the art. See e.g., Sambrook et al., Molecular Cloning: A Laboratory Manual, 4^{th} Ed., Cold Spring Harbor Press, Cold Spring Harbor, N.Y., 2012. Other techniques include digital, multiplexed analysis of gene expression, such as the nCounter® (NanoString Technologies, Seattle, WA) gene expression assays, which are further described in US20100112710 and US20100047924.

Detecting a nucleic acid of interest generally involves hybridization between a target (e.g. mRNA or cDNA) and a probe. The nucleic acid sequences of the genes and gene mutations described herein are known. Therefore, one of skill in the art can readily design hybridization probes for detecting those genes. See, e.g., Sambrook et al., Molecular Cloning: A Laboratory Manual, 4^{th} Ed., Cold Spring Harbor Press, Cold Spring Harbor, N.Y., 2012. Each probe may be substantially specific for its target, to avoid any cross-hybridization and false positives. An alternative to using specific probes is to use specific reagents when deriving materials from transcripts (e.g., during cDNA production, or using target-specific primers during amplification). In both cases specificity can be achieved by hybridization to portions of the targets that are substantially unique within the group of genes being analyzed, for example hybridization to the polyA tail would not provide specificity. If a target has multiple splice variants, it is possible to design a hybridization reagent that recognizes a region common to each variant and/or to use more than one reagent, each of which may recognize one or more variants. For example, polynucleotide probes that specifically bind to the mRNA transcripts of the genes described herein (or cDNA synthesized therefrom) can be created using the nucleic acid sequences of the mRNA or cDNA targets themselves by routine techniques (e.g., PCR or synthesis).

RNA-sequencing (RNA-seq) may be used to detect a nucleic acid of interest. As used herein, RNA-seq, also called Whole Transcriptome Shotgun Sequencing, refers to any of a variety of high-throughput sequencing techniques used to detect the presence and quantity of RNA transcripts in real time. See Wang, Z., M. Gerstein, and M. Snyder, RNA-Seq: a revolutionary tool for transcriptomics, NAT REV GENET, 2009. 10(1): p. 57-63. RNA-seq can be used to reveal a snapshot of a sample’s RNA from a genome at a given moment in time. RNA can be converted to cDNA fragments via reverse transcription prior to sequencing, or RNA can be directly sequenced from RNA fragments without conversion to cDNA. Adaptors may be attached to the 5′ and/or 3′ ends of the fragments, and the RNA or cDNA may optionally be amplified, for example by PCR. The fragments are then sequenced using high-throughput sequencing technology, such as, for example, those available from Roche (e.g., the 454 platform), Illumina, Inc., and Applied Biosystem (e.g., the SOLiD system).

Microarray analysis or a PCR-based method may also be used to detect a nucleic acid of interest, including, but not limited to, real-time PCR, nested PCT, quantitative PCR, multiplex PCR, and droplet digital PCR. In this respect, measuring the expression of the foregoing nucleic acids in a biological sample can comprise, for instance, contacting a sample with polynucleotide probes specific to the genes of interest, or with primers designed to amplify a portion of the genes of interest, and detecting binding of the probes to the nucleic acid targets or amplification of the nucleic acids, respectively. Detailed protocols for designing PCR primers are known in the art. See e.g., Sambrook et al., Molecular Cloning: A Laboratory Manual, 4^{th} Ed., Cold Spring Harbor Press, Cold Spring Harbor, N.Y., 2012. Similarly, detailed protocols for preparing and using microarrays to analyze gene expression and gene mutations are known in the art and described herein.

Stringency of hybridization reactions is readily determinable by one of ordinary skill in the art, and generally is an empirical calculation dependent upon probe length, washing temperature, and salt concentration. In general, longer probes may require higher temperatures for proper annealing, while shorter probes may require lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so.

“Stringent conditions” or “high stringency conditions,” as defined herein, are identified by, but not limited to, those that: (1) use low ionic strength and high temperature for washing, for example 0.015 M sodium chloride/0.0015 M sodium citrate/0.1% sodium dodecyl sulfate at 50° C.; (2) use during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) use 50% formamide, 5XSSC (0.75 M NaCl, 0.075 M sodium citrate), 50 mM sodium phosphate (pH 6.8), 0.1% sodium pyrophosphate, 5X Denhardt’s solution, sonicated salmon sperm DNA (50 µg/ml), 0.1% SDS, and 10% dextran sulfate at 42° C., with washes at 42° C. in 0.2XSSC (sodium chloride/sodium citrate) and 50% formamide at 55° C., followed by a high-stringency wash of 0.1XSSC containing EDTA at 55° C. “Moderately stringent conditions” are described by, but not limited to, those in Sambrook et al., Molecular Cloning: A Laboratory Manual, New York: Cold Spring Harbor Press, 1989, and include the use of washing solution and hybridization conditions (e.g., temperature, ionic strength and % SDS) less stringent than those described above. An example of moderately stringent conditions is overnight incubation at 37° C. in a solution comprising: 20% formamide, 5XSSC (150 mM NaCl, 15 mM trisodium citrate), 50 mM sodium phosphate (pH 7.6), 5X Denhardt’s solution, 10% dextran sulfate, and 20 mg/mL denatured sheared salmon sperm DNA, followed by washing the filters in 1XSSC at about 37-50° C. The skilled artisan will recognize how to adjust the temperature, ionic strength, etc. as necessary to accommodate factors such as probe length and the like.

RNA obtained from a sample may be subjected to qRT-PCR. Reverse transcription may occur by any methods known in the art, such as through the use of an Omniscript RT Kit (Qiagen). The resultant cDNA may then be amplified by any amplification technique known in the art. Gene expression or gene mutation may then be analyzed through the use of, for example, control samples. Detailed protocols for preparing and using microarrays to analyze gene expression and gene mutations are known in the art and described herein.

Alternatively or additionally, gene mutations and gene expression levels can be determined at the protein level, meaning that levels of proteins encoded by the genes discussed herein are measured. Several methods and devices are known for determining levels of proteins including immunoassays, such as described, for example, in U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; 5,458,852; and 5,480,792, each of which is hereby incorporated by reference in its entirety. These assays may include various sandwich, competitive, or non-competitive assay formats, to generate a signal that is related to the presence or amount of a protein of interest. Any suitable immunoassay may be utilized, for example, lateral flow, enzyme-linked immunoassays (ELISA), radioimmunoassays (RIAs), competitive binding assays, and the like. Numerous formats for antibody arrays have been described. Such arrays may include different antibodies having specificity for different proteins intended to be detected. For example, at least 100 different antibodies are used to detect 100 different protein targets, each antibody being specific for one target. Other ligands having specificity for a particular protein target can also be used, such as the synthetic antibodies disclosed in WO 2008/048970, which is hereby incorporated by reference in its entirety. Other compounds with a desired binding specificity can be selected from random libraries of peptides or small molecules. U.S. Pat. No. 5,922,615, which is hereby incorporated by reference in its entirety, describes a device that uses multiple discrete zones of immobilized antibodies on membranes to detect multiple target antigens in an array. Microtiter plates or automation can be used to facilitate detection of large numbers of different proteins.

One type of immunoassay, called nucleic acid detection immunoassay (NADIA), combines the specificity of protein antigen detection by immunoassay with the sensitivity and precision of the polymerase chain reaction (PCR). This amplified DNA-immunoassay approach is similar to that of an enzyme immunoassay, involving antibody binding reactions and intermediate washing steps, except the enzyme label is replaced by a strand of DNA and detected by an amplification reaction using an amplification technique, such as PCR. Exemplary NADIA techniques are described in U.S. Pat. No. 5,665,539 and published U.S. Application 2008/0131883, both of which are hereby incorporated by reference in their entirety. Briefly, NADIA uses a first (reporter) antibody that is specific for the protein of interest and labelled with an assay-specific nucleic acid. The presence of the nucleic acid does not interfere with the binding of the antibody, nor does the antibody interfere with the nucleic acid amplification and detection. Typically, a second (capturing) antibody that is specific for a different epitope on the protein of interest is coated onto a solid phase (e.g., paramagnetic particles). The reporter antibody/nucleic acid conjugate is reacted with sample in a microtiter plate to form a first immune complex with the target antigen. The immune complex is then captured onto the solid phase particles coated with the capture antibody, forming an insoluble sandwich immune complex. The microparticles are washed to remove excess, unbound reporter antibody/nucleic acid conjugate. The bound nucleic acid label is then detected by subjecting the suspended particles to an amplification reaction (e.g. PCR) and monitoring the amplified nucleic acid product.

Although immunoassays have been used for the identification and quantification of proteins, recent advances in mass spectrometry (MS) techniques have led to the development of sensitive, high-throughput MS protein analyses. The MS methods can be used to detect low abundant proteins in complex biological samples. For example, it is possible to perform targeted MS by fractionating the biological sample prior to MS analysis. Common techniques for carrying out such fractionation prior to MS analysis include, for example, two-dimensional electrophoresis, liquid chromatography, and capillary electrophoresis. Selected reaction monitoring (SRM), also known as multiple reaction monitoring (MRM), has also emerged as a useful high-throughput MS-based technique for quantifying targeted proteins in complex biological samples, including prostate cancer biomarkers that are encoded by gene fusions (e.g., TMPRSS2/ERG).

The presence or absence of a gene mutation may also be determined by whole genome sequencing of one or more cells in a biological sample, wherein the sequence of the target genes are analysed for mutations. Gene mutations that may be detected through the methods disclosed herein include deleterious mutations such as missense changes, nonsense changes, and genomic rearrangements; copy number variants, decreased or absent expression levels, such as mRNA expression or protein expression, and methylation patterns that indicate decreased or absent expression levels. In certain embodiments of all aspects of the present disclosure, a gene mutation may be a pathogenic (P) gene mutation, and in certain embodiments of all aspects of the present disclosure, a gene mutation may be a likely pathogenic (LP) gene mutation. In certain embodiments of all aspects of the present disclosure, a gene mutation may be considered a pathogenic/likely pathogenic (P/LP) gene mutation.

### Biological Samples

The methods described herein involve analysis of germline mutations in biological samples obtained from a patient, such as a patient who has been diagnosed with prostate cancer or a patient who is at risk of being diagnosed with prostate cancer, based, for example, on family history. The biological sample may comprise prostate tissue and can be obtained through a biopsy, such as a transrectal or transperineal biopsy. Alternatively, the biological sample may comprise prostate tissue obtained from radical prostatectomy. Biological samples may include cancer cells or non-cancer cells. Cancer cells may be found in a biological sample, such as a tumor, a tissue, or blood. In certain embodiments of all aspects of the present disclosure, the biological sample comprises non-cancer somatic cells taken from any body tissue or fluid, such as blood or blood derivatives (serum, plasma, etc.), saliva, semen or seminal fluid, urine, or cerebrospinal fluid.

Urine samples may be collected following a digital rectal examination (DRE) or a prostate biopsy. The sample may also contain tumor-derived exosomes. Exosomes are small (typically 30 to 100 nm) membrane-bound particles that are released from normal, diseased, and neoplastic cells and are present in blood and other bodily fluids.

Nucleic acids or polypeptides may be isolated from the sample prior to detecting a germline mutation. The methods disclosed herein can be used with biological samples collected from a variety of mammals, and in certain embodiments, the methods disclosed herein may be used with biological samples obtained from a human subject. In some embodiments of all aspects of the present disclosure, the biological sample may be obtained from a patient of African descent. In some embodiments of all aspects of the present disclosure, the biological sample may be obtained from a patient that has not been diagnosed with prostate cancer. In some embodiments of all aspects of the present disclosure, the biological sample may be obtained from a patient who has a family history of cancer or a family history of DDRG germline mutation related cancer.

### Prostate Cancer

This application discloses certain gene panels that are associated with prostate cancer, wherein at least one of the genes in the gene panel may contain a germline mutation that is a pathogenic or likely pathogenic mutation. Detecting a germline mutation in a target gene or genes in a biological sample can be used to identify a patient as being at an increased risk for developing prostate cancer, or for diagnosing or prognosing a patient with prostate cancer. The presence of a germline mutation of a gene in the gene panel may also be used to measure the severity or aggressiveness of prostate cancer, for example, distinguishing between well-differentiated prostate cancer and poorly-differentiated prostate cancer and/or identifying prostate cancer that has metastasized or recurred following prostatectomy or is more likely to metastasize or recur following prostatectomy. The presence of a germline mutation of certain genes in the gene panel indicates that a patient, such as a patient of African descent, is at an increased risk of experiencing a biochemical recurrence of a cancer, such as prostate cancer.

Prostate cancer may, in certain instances, be hereditary. Germline mutations have been found to be present in approximately 12% of patients diagnosed with metastatic prostate cancer. Gomella, et al., Introduction to the 2019 Philadelphia Prostate Cancer Consensus Program: ‘Implementation of Genetic Testing for Inherited Prostate Cancer’, Canadian J. of Urol. 2019; 26:1-4. Early diagnosis of prostate cancer can lead to significant improvement in survival outcomes. Therefore, if an individual can be determined to have a predisposition to developing hereditary prostate cancer based on the presence of certain germline mutations before cancer develops, then early surveillance, screening, and preventative measures could lead to early diagnosis and improved prognosis. Accordingly, the patient may be one who has never been diagnosed with prostate cancer and has a family history of cancer, such as a family history of prostate cancer or breast cancer. Alternatively, the patient may be one who has never been diagnosed with prostate cancer and does not have a family history of cancer.

When prostate cancer is found in a biopsy, it is typically graded to estimate how quickly it is likely to grow and spread. The most commonly used prostate cancer grading system, called Gleason grading, evaluates prostate cancer cells on a scale of 1 to 5, based on their pattern when viewed under a microscope. Cancer cells that still resemble healthy prostate cells have uniform patterns with well-defined boundaries and are considered well-differentiated (Gleason grades 1 and 2). The more closely the cancer cells resemble prostate tissue, the more the cells will behave like normal prostate tissue and the less aggressive the cancer. Gleason grade 3, the most common grade, shows cells that are moderately differentiated, that is, still somewhat well-differentiated, but with boundaries that are not as well-defined. Poorly-differentiated cancer cells have random patterns with poorly defined boundaries and no longer resemble prostate tissue (Gleason grades 4 and 5), indicating a more aggressive cancer.

Prostate cancers often have areas with different grades. A combined Gleason score is determined by adding the grades from the two most common cancer cell patterns within the tumor. For example, if the most common pattern is grade 4 and the second most common pattern is grade 3, then the combined Gleason score is 4+3=7. If there is only one pattern within the tumor, the combined Gleason score can be as low as 1+1=2 or as high as 5+5=10. Combined scores of 2 to 4 are considered well-differentiated, scores of 5 to 6 are considered moderately-differentiated and scores of 7 to 10 are considered poorly-differentiated. Cancers with a high Gleason score are more likely to have already spread beyond the prostate gland (metastasized) at the time they were found.

In general, the lower the Gleason score, the less aggressive the cancer and the better the prognosis (outlook for cure or long-term survival). The higher the Gleason score, the more aggressive the cancer and the poorer the prognosis for long-term, metastasis-free survival.

In the methods described herein, a sample is assayed to determine if a germline mutation is present in each gene of the specified gene panel. Detecting a germline mutation in at least one gene of the gene panels described herein can indicate that a patient, who has not previously been diagnosed with prostate cancer, is at an increased risk of developing prostate cancer in the future. Alternatively, the patient at an increased risk of developing prostate cancer in the future has a family history of prostate cancer Detecting a germline mutation in at least one gene of the gene panels described herein can indicate that a prostate cancer has an increased risk of metastasizing, particularly in human subjects of African descent. For example, a germline mutation in at least one of the following 5 genes can indicate that a patient of African descent is at an increased risk of developing prostate cancer in the future: BRCA1, PMS2, RAD51, RAD54B, and RAD54L. In another aspect disclosed herein, detecting a germline mutation in at least one of the following 13 genes can indicate that a patient of African descent is at an increased risk of developing prostate cancer in the future: BRCA1, CHEK2, ERCC2, FANCA, FANCL, MSH6, MUTYH, OGG1, PMS2, POLG, RAD51, RAD54B, and RAD54L. In yet another aspect disclosed herein, detecting a germline mutation in at least one of the following 8 genes can indicate that a patient of African descent is at an increased risk of developing prostate cancer in the future: BRCA1, FANCA, FANCL, MSH6, PMS2, RAD51, RAD54B, and RAD54L.

### Gene Mutation Profiles

The gene panels and gene mutation profiles disclosed herein may be used to identify or characterize prostate cancer in a subject, such as a human subject of African descent. For example, when a sample is assayed to determine if it contains a germline mutation in each of the genes in a gene panel, as described herein, such as the 5-gene panel, the 16-gene panel and the 8-gene panel, a germline mutation of at least one gene in the gene panel may be detected and used to identify a subject, such as a human subject of African descent, as being at a high risk for developing prostate cancer in the future. Likewise, the absence of a germline mutation in any genes of the gene panel as disclosed herein may be used to identify a subject, such as a human subject of African descent, as being at a low risk for developing prostate cancer in the future.

In certain embodiments of all aspects of the present disclosure, the germline mutation that may be detected is chosen from a mutation in at least one of the following 5 DDRG genes: BRCA1, PMS2, RAD51, RAD54B, and RAD54L.

In certain embodiments of all aspects of the present disclosure, the germline mutation that may be detected is chosen from a mutation in at least one of the following 47 DDRG genes: ATM, ATR, BLM, BRCA1, BRCA2, CHEK2, DNA2, ERCC2, ERCC3, ERCC4, ERCC6, FAN1, FANCA, FANCC, FANCD2, FANCI, FANCL, GTF2H5, HFM1, IDH1, IN080, LIG1, MLH3, MSH2, MSH6, MUTYH, NBN, NTHL1, OGG1, PCNA, PMS2, PNKP, POLG, POLH, POLK, RAD51, RAD51C, RAD54B, RAD54L, RRM2B, TDP2, TELO2, TP53, TTK, TUBGCP4, UNG, and XPA.

In certain embodiments of all aspects of the present disclosure, the germline mutation that may be identified is chosen from a mutation in at least one of the following 25 DDRG genes: ATM, BRCA1, BRCA2, CHEK2, ERCC2, FAN1, FANCA, FANCC, FANCD2, FANCI, FANCL, GTF2H5, MLH3, MSH2, MSH6, MUTYH, NBN, OGG1, PMS2, POLG, POLH, RAD51, RAD54B, RAD54L, and RAD51C.

In certain embodiments of all aspects of the present disclosure, the germline mutation that may be identified is chosen from a mutation in at least one of the following 19 DDRG genes: ATM, BRCA1, CHEK2, ERCC2, FAN1, FANCA, FANCD2, FANCL, GTF2H5, MSH6, MUTYH, NBN, OGG1, PMS2, POLG, POLH, RAD51, RAD54B, and RAD54L.

In certain embodiments of all aspects of the present disclosure, the germline mutation that may be identified is chosen from a mutation in at least one of the following 16 DDRG genes: BRCA1, BRCA2, FAN1, FANCA, FANCC, FANCD2, FANCI, FANCL, MLH, MSH2, MSH6, PMS2, RAD51, RAD51C, RAD54B, and RAD54L. In certain embodiments, each of the DDRG genes in the 16-gene panel is targetable by at least one PARP inhibitor, and in certain embodiments, the germline mutation is present in a biological sample from a patient of African descent.

In certain embodiments of all aspects of the present disclosure, the germline mutation that may be identified is chosen from a mutation in at least one of the following 13 DDRG genes: BRCA1, CHEK2, ERCC2, FANCA, FANCL, MSH6, MUTYH, OGG1, PMS2, POLG, RAD51, RAD54B, and RAD54L.

In certain embodiments of all aspects of the present disclosure, the germline mutation that may be identified is chosen from a mutation in at least one of the following 8 DDRG genes. BRCA1, FANCA, FANCL, MSH6, PMS2, RAD51, RAD54B, and RAD54L.

In certain embodiments of all aspects of the present disclosure, the germline mutation that may be detected is chosen from a mutation in at least one of the following 12 DDRG genes: FANCA, MUTYH, OGG1, MSH6, POLG, RAD51, FANCL, RAD54L, CHEK2, POLH, NBN, and TELO2. In certain embodiments, each of the DDRG genes in the 8-gene panel is targetable by at least one PARP inhibitor, and in certain embodiments, the germline mutation is present in a biological sample from a patient of African descent.

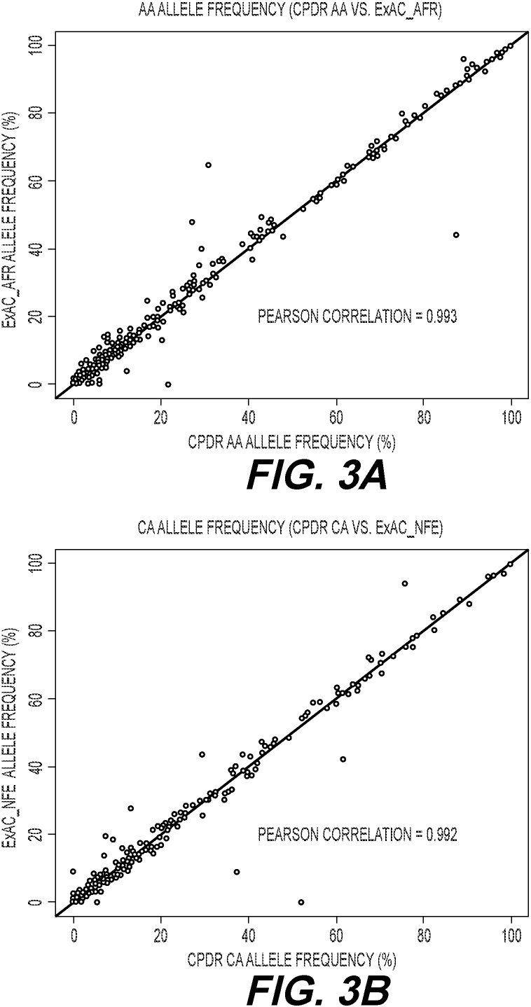

In certain embodiments of all aspects of the present disclosure, including any of the 5-gene panel, the 47-gene panel, the 25-gene panel, the 19-gene panel, the 16-gene panel, the 13-gene panel or the 8-gene panel, a germline mutation that may be identified is further chosen from a mutation in MDC1. MDC1 is a known prostate cancer suppressor gene that is believed to be responsible for co-activating androgen receptors and acting as an androgen receptor-induced transactivator. See FIG. 4.

### Controls

The control may be any suitable reference that allows evaluation of the nucleotide sequence of genes or the expression level of the genes in the biological sample as compared to the nucleotide sequence or the expression of the same genes in a sample comprising control cells. For example, the control cells may be somatic cells obtained from a patient or pool of patients who have never been diagnosed with cancer, including prostate cancer, and who do not have a family history of cancer, including prostate cancer. Thus, for instance, the control can be a sample that is analyzed simultaneously or sequentially with the test sample. The control can also be embodied, for example, in data that reflects the sequences of the target genes in a sample or pool of samples known to contain wild-type sequences of those target genes, such as might be part of an electronic database or computer program, such as those available from the Exome Aggregation Consortium (ExAc) or the Genome Aggregation Database (gnomAD).

The control may also be a predetermined “cut-off” or threshold value of absolute expression. Thus, the control can be embodied, for example, in a pre-prepared microarray used as a standard or reference, or in data that reflects the expression profile of relevant gene mutations in a sample or pool of samples that do not contain the gene mutations, such as might be part of an electronic database or computer program.

### Arrays

A convenient way of measuring RNA transcript levels for multiple genes in parallel is to use an array (also referred to as microarrays in the art). A useful array may include multiple polynucleotide probes (such as DNA) that are immobilized on a solid substrate (e.g., a glass support such as a microscope slide, or a membrane) in separate locations (e.g., addressable elements) such that detectable hybridization can occur between the probes and the transcripts to indicate the amount of each transcript that is present. The arrays disclosed herein can be used to detect mutations in the genes of the gene panel disclosed herein.

For example, in the 5-gene panel, the array may comprise (a) a substrate and (b) at least 5, such as at least 6, at least 8, at least 9, or at least 10 different addressable elements that each comprise at least one polynucleotide probe for detecting expression of an mRNA transcript (or cDNA synthesized from the mRNA transcript) that is specific for a gene mutation in one of the genes in the 5-gene panel, such that the array can be used to simultaneously detect at least one gene mutation in at least 5 of the genes in the gene panels disclosed herein. In another example, in 47-gene panel, the array may comprises (a) a substrate and (b) at least 5, such as at least 6, at least 8, at least 9, at least 10, at least 13, at least 15, at least 16, at least 19, at least 20, at least 25, at least 30, at least 35, at least 40, at least 45, or 47 different addressable elements that each comprise at least one polynucleotide probe for detecting the expression of an mRNA transcript (or cDNA synthesized from the mRNA transcript) that is specific for a gene mutation in one of the genes in the 47-gene panel, such that the array can be used to simultaneously detect at least one gene mutation in at least 5, at least 6, at least 8, at least 9, at least 10, at least 13, at least 15, at least 16, at least 19, at least 20, at least 25, at least 30, at least 35, at least 40, at least 45, or 47 of the genes in the gene panels disclosed herein.

In one embodiment of all aspects of the disclosure, the substrate comprises at least 5, such as at least 6, at least 8, at least 9, at least 10, at least 13, at least 15, at least 16, at least 19, at least 20, or 25 different addressable elements, wherein each different addressable element is specific for one of the genes in the 25-gene panel, such that the array can be used to simultaneously detect at least one gene mutation in at least at 5, at least 6, at least 8, at least 9, at least 10, at least 13, at least 15, at least 16, at least 19, at least 20, or 25 of the genes in the gene panels described herein.

In another embodiment of all aspects of the disclosure, the substrate comprises at least 5, such as at least 6, at least 8, at least 9, at least 10, at least 13, at least 15, at least 16, or 19 different addressable elements, wherein each different addressable element is specific for one of the genes in the 19-gene panel, such that the array can be used to simultaneously detect at least one gene mutation in at least 5, at least 6, at least 8, at least 9, at least 10, at least 13, at least 15, at least 16, or 19 of the genes in the gene panels described herein.

In another embodiment of all aspects of the disclosure, the substrate comprises at least 5, such as at least 6, at least 8, at least 9, at least 10, at least 13, or 16 different addressable elements, wherein each different addressable element is specific for one of the genes in the 16-gene panel, such that the array can be used to simultaneously detect at least one gene mutation in at least 5, at least 6, at least 8, at least 9, at least 10, at least 13, or 16 of the genes in the gene panels described herein.

In another embodiment of all aspects of the disclosure, the substrate comprises at least 5, such as at least 6, at least 8, at least 9, at least 10, or 13 different addressable elements, wherein each different addressable element is specific for one of the genes in the 13-gene panel, such that the array can be used to simultaneously detect at least one gene mutation in at least 5, at least 6, at least 8, at least 9, at least 10, or 13 of the genes in the gene panels described herein.

In another embodiment of all aspects of the disclosure, the substrate comprises at least 5, such as at least 6, at least 7, or 8 different addressable elements, wherein each different addressable element is specific for one of the genes in the 8-gene panel, such that the array can be used to simultaneously detect at least one gene mutation in at least 5, at least 6, at least 7, or 8 of the genes in the gene panels described herein.

The array can also further comprises one or more different addressable elements comprising at least one oligonucleotide probe for detecting the expression of an mRNA transcript (or cDNA synthesized from the mRNA transcript) of a control gene.

As used herein, the term “addressable element” means an element that is attached to the substrate at a predetermined position and specifically binds a known target molecule, such that when target-binding is detected (e.g., by fluorescent labeling), information regarding the identity of the bound molecule is provided on the basis of the location of the element on the substrate. Addressable elements are “different” for the purposes of the present disclosure if they do not bind to the same target gene. The addressable element comprises one or more polynucleotide probes specific for an mRNA transcript of a given gene, or a cDNA synthesized from the mRNA transcript. The addressable element can comprise more than one copy of a polynucleotide or can comprise more than one different polynucleotide, provided that all of the polynucleotides bind the same target molecule. Where a gene is known to express more than one mRNA transcript, the addressable element for the gene can comprise different probes for different transcripts, or probes designed to detect a nucleic acid sequence common to two or more (or all) of the transcripts. Alternatively, the array can comprise an addressable element for the different transcripts. The addressable element also can comprise a detectable label, suitable examples of which are well known in the art.

The array can comprise addressable elements that bind to mRNA or cDNA other than that of the above-referenced 47 genes or a subset of the above-referenced 47 genes (such as 25 genes, 19 genes, 16, genes, 13 genes, 8 genes or 5 genes). However, an array capable of detecting a vast number of targets (e.g., mRNA or polypeptide targets), such as arrays designed for comprehensive expression profiling of a cell line, chromosome, genome, or the like, may not be economical or convenient for collecting data to use in diagnosing and/or prognosing cancer. Thus, the array typically comprises no more than about 1000 different addressable elements, such as no more than about 500 different addressable elements, no more than about 250 different addressable elements, or even no more than about 100 different addressable elements, such as about 75 or fewer different addressable elements, about 60 or fewer different addressable elements, about 50 or fewer different addressable elements, about 47 or fewer different addressable elements, about 40 or fewer different addressable elements, about 35 or fewer addressable elements, about 30 or fewer different addressable elements, about 25 or fewer, about 20 or fewer, about 19 or fewer, about 16 or fewer, about 15 or fewer, about 13 or fewer, about 10 or fewer, about 9 or fewer, about 8 or fewer, about 6 or fewer, or about 5 different addressable elements.

It is also possible to distinguish these diagnostic arrays from the more comprehensive genomic arrays and the like by limiting the number of polynucleotide probes on the array. Thus, the array typically has polynucleotide probes for no more than 1000 genes immobilized on the substrate. The array can also have oligonucleotide probes for no more than 500, no more than 250, no more than 100, no more than 75, no more than 60, or no more than 50 genes. Or the array can have oligonucleotide probes for no more than 47 genes, no more than 40 genes, no more than 35 genes, no more than 30 genes, no more than 25 genes, no more than 20 genes, no more than 19 genes, no more than 16 genes, no more than 15 genes, no more than 13 genes, no more than 10 genes, no more than 9 genes, no more than 8 genes, no more than 6 genes, or no more than 5 genes.

The substrate can be any rigid or semi-rigid support to which polynucleotides can be covalently or non-covalently attached. Suitable substrates include membranes, filters, chips, slides, wafers, fibers, beads, gels, capillaries, plates, polymers, microparticles, and the like. Materials that are suitable for substrates include, for example, nylon, glass, ceramic, plastic, silica, aluminosilicates, borosilicates, metal oxides such as alumina and nickel oxide, various clays, nitrocellulose, and the like.

The polynucleotides of the addressable elements (also referred to as “probes”) can be attached to the substrate in a pre-determined 1- or 2-dimensional arrangement, such that the pattern of hybridization or binding to a probe is easily correlated with the expression of a particular gene. Because the probes are located at specified locations on the substrate (i.e., the elements are “addressable”), the hybridization or binding patterns and intensities create a unique expression profile, which can be interpreted in terms of expression levels of particular genes and can be correlated with prostate cancer in accordance with the methods described herein.

The array can comprise other elements common to polynucleotide arrays. For instance, the array also can include one or more elements that serve as a control, standard, or reference molecule, such as a housekeeping gene or portion thereof, to assist in the normalization of expression levels or the determination of nucleic acid quality and binding characteristics, reagent quality and effectiveness, hybridization success, analysis thresholds and success, etc. These other common aspects of the arrays or the addressable elements, as well as methods for constructing and using arrays, including generating, labeling, and attaching suitable probes to the substrate, consistent with the invention are well-known in the art. Other aspects of the array are as described with respect to the methods disclosed herein.

An array can also be used to measure protein levels of multiple proteins in parallel. Such an array comprises one or more supports bearing a plurality of ligands that specifically bind to a plurality of proteins, wherein the plurality of proteins comprises no more than 500, no more than 250, no more than 100, no more than 75, no more than 60, no more than 50, no more than 47, no more than 45, no more than 40, no more than 35, no more than 30, no more than 25, no more than 20, no more than 19, no more than 16, no more than 15, no more than 13, no more than 10, no more than 9, no more than 8, no more than 6, or no more than 5 different proteins. The ligands are optionally attached to a planar support or beads. Typically, the ligands are antibodies. However, any ligand that specifically binds to a protein of interest may be used. The proteins that are to be detected using the array correspond to the proteins encoded by the nucleic acids of interest, as described above, including the specific gene panels disclosed. Thus, each ligand (e.g. antibody) is designed to bind to one of the target proteins (e.g., polypeptide sequences encoded by the genes disclosed herein). As with the nucleic acid arrays, each ligand may be associated with a different addressable element to facilitate detection of the different proteins in a sample.

Sequencing methods, including but not limited to, next-generation sequencing (NGS) techniques, can also be used to detect one or more gene mutations of interest. For example, disclosed herein are methods of obtaining a gene mutation profile in a biological sample, the method comprising: a) sequencing genes of interest in a biological sample; and b) detecting the presence or absence of a pathogenic or likely pathogen mutation in the genes of interest.

### Methods of Predicting, Diagnosing, or Prognosing Cancer

Disclosed herein are methods of predicting, diagnosing, or prognosing prostate cancer in a patient, the method comprising (1) testing a biological sample from the patient for the presence or absence of germline mutations in the genes of a gene panel; and (2) identifying the patient as having a high risk for prostate cancer if the presence of a germline mutation in a gene is detected.

In certain embodiments of all aspects of the disclosure, testing a biological sample from the patient comprises determining the presence or absence of a germline mutation of a plurality of genes in the biological sample, wherein the plurality of genes comprises at least 5, such as at least 6, at least 8, at least 9, at least 10, at least 13, at least 15, at least 16, at least 19, at least 20, at least 25, at least 30, at least 35, at least 40, at least 45, or 47 of the following genes in the 47-gene panel: ATM, ATR, BLM, BRCA1, BRCA2, CHEK2, DNA2, ERCC2, ERCC3, ERCC4, ERCC6, FAN1, FANCA, FANCC, FANCD2, FANCI, FANCL, GTF2H5, HFM1, IDH1, IN080, LIG1, MLH3, MSH2, MSH6, MUTYH, NBN, NTHL1, OGG1, PCNA, PMS2, PNKP, POLG, POLH, POLK, RAD51, RAD51C, RAD54B, RAD54L, RRM2B, TDP2, TELO2, TP53, TTK, TUBGCP4, UNG, and XPA.

In certain embodiments of all aspects of the disclosure, testing a biological sample from the patient comprises determining the presence or absence of a germline mutation in a plurality of genes in the biological sample, wherein the plurality of genes comprises at least 5, such as at least 6, at least 8, at least 9, at least 10, at least 13, at least 15, at least 16, at least 19, or 25 of the following genes in the 25-gene panel: ATM, BRCA1, BRCA2, CHEK2, ERCC2, FAN1, FANCA, FANCC, FANCD2, FANCI, FANCL, GTF2H5, MLH3, MSH2, MSH6, MUTYH, NBN, OGG1, PMS2, POLG, POLH, RAD51, RAD54B, RAD54L, and RAD51C. For example, the plurality of genes may comprise at least 8 of the genes in the 25-gene panel, including, for example, ATM, BRAC1, BRAC2, CHEK2, MSH2, MSH6, NBN, and PMS2.

In certain embodiments of all aspects of the disclosure, testing a biological sample from the patient comprises determining the presence or absence of a germline mutation in a plurality of genes in the biological sample, wherein the plurality of genes comprises at least 5, such as at least 6, at least 8, at least 9, at least 10, at least 13, at least 15, at least 16, or 19 of the following genes in the 19-gene panel: ATM, BRCA1, CHEK2, ERCC2, FAN1, FANCA, FANCD2, FANCL, GTF2H5, MSH6, MUTYH, NBN, OGG1, PMS2, POLG, POLH, RAD51, RAD54B, and RAD54L.

In certain embodiments of all aspects of the disclosure, testing a biological sample from the patient comprises determining the presence or absence of a germline mutation in a plurality of genes in the biological sample, wherein the plurality of genes comprises at least 5, such as at least 6, at least 8, at least 9, at least 10, at least 13, at least 15, or 16 of the following genes in the 16-gene panel: BRCA1, BRCA2, FAN1, FANCA, FANCC, FANCD2, FANCI, FANCL, MLH, MSH2, MSH6, PMS2, RAD51, RAD51C, RAD54B, and RAD54L.

In certain embodiments of all aspects of the disclosure, testing a biological sample from the patient comprises determining the presence or absence of a germline mutation in a plurality of genes in the biological sample, wherein the plurality of genes comprises at least 5, such as at least 6, at least 8, at least 9, at least 10, or 13 of the following genes in the 13-gene panel: BRCA1, CHEK2, ERCC2, FANCA, FANCL, MSH6, MUTYH, OGG1, PMS2, POLG, RAD51, RAD54B, and RAD54L.

In certain embodiments of all aspects of the disclosure, testing a biological sample from the patient comprises determining the presence or absence of a germline mutation in a plurality of genes in the biological sample, wherein the plurality of genes comprises at least 5, such as at least 7, at least 6, or 8 of the following genes in the 8-gene panel: BRCA1, FANCA, FANCL, MSH6, PMS2, RAD51, RAD54B, and RAD54L.

In certain embodiments of the disclosure, a patient may be identified as having a high risk of developing prostate cancer by determining the presence of a germline mutation in at least one gene in the 5-gene panel. In certain embodiments of the disclosure, a patient may be identified as having a high risk of developing prostate cancer by determining the presence of a germline mutation in at least one gene in the 47-gene panel. In certain embodiments, a patient may be identified as having a high risk of developing prostate cancer by determining the presence of a germline mutation in at least one gene in the 25-gene panel. In certain embodiments, a patient may be identified as having a high risk of developing prostate cancer by determining the presence of a germline mutation in at least one gene in the 19-gene panel. In certain embodiments, a patient may be identified as having a high risk of developing prostate cancer by determining the presence of a germline mutation in at least one gene in the 16-gene panel. In certain embodiments, a patient may be identified as having a high risk of developing prostate cancer by determining the presence of a germline mutation in at least one gene in the 13-gene panel. In certain embodiments, a patient may be identified as having a high risk of developing prostate cancer by determining the presence of a germline mutation in at least one gene in the 8-gene panel.

In certain embodiments, a patient may be identified as having a high risk of developing prostate cancer if the patient is of African descent and if the presence of a germline mutation in at least one gene in the 5-gene panel is found. In certain embodiments, a patient may be identified as having a high risk of developing prostate cancer if the patient is of African descent and if the presence of a germline mutation in at least one gene in the 16-gene panel is found. In certain embodiments, a patient may be identified as having a high risk of developing prostate cancer if the patient is of African descent and if the presence of a germline mutation in at least one gene in the 8-gene panel is found. In certain embodiments, the patient has a family history of cancer, such as prostate or breast cancer. In certain embodiments, the plurality of genes in the gene panel, such as the 5-gene, 47-gene, 25-gene, 19-gene, 16-gene, 13-gene, or 8-gene panel, further comprises MDC1.

### Genetic Testing

In some embodiments of all aspects of the present disclosure, there is a genetic testing method for identifying a patient having a predisposition for developing prostate cancer, the method comprising obtaining a biological sample from the patient and assaying the biological sample to determine if the biological sample contains at least one germline mutation from a plurality of human genes, wherein the plurality of human genes comprises BRCA1, PMS2, RAD51, RAD54B, and RAD54L. In some embodiments of all aspects of the present disclosure, there is a genetic testing method for identifying a patient having a predisposition for developing prostate cancer, the method comprising obtaining a biological sample from the patient and assaying the biological sample to determine if the biological sample contains at least one germline mutation from a plurality of human genes, wherein the plurality of human genes comprises: at least 15, such as at least 20, at least 25, at least 30, at least 35, at least 40, at least 45, or all 47 genes of the 47-gene panel; at least 13, such as at least 15, at least 20, or all 25 genes of the 25-gene panel; at least 10, such as at least 15, or all 19 genes of the 19-gene panel; at least 9, such as at least 10, at least 15, or all 16 genes of the 16-gene panel; at least 8, such as at least 10, or all 13 genes of the 13-gene panel; or at least 6, such as at least 7, or all 8 genes of the 8-gene panel, wherein the patient is identified as having a predisposition for developing prostate cancer if at least one germline mutation is detected in at least one of the at least 15 human genes. In certain embodiments, prior to assaying the biological sample, the patient is identified as having a family history of cancer, such as a family history of DDRG germline mutation related cancer, including prostate cancer or breast cancer or ovarian cancer or colorectal cancer. In certain embodiments, the patient is of African descent.

### Assessment of Disease Severity and Aggressiveness of Prostate Cancer

In some embodiments of all aspects of the present disclosure, there is a method of characterizing prostate cancer in a patient comprising assaying a biological sample obtained from the patient to determine if the biological sample contains at least one germline mutation in a plurality of human genes, wherein the plurality of human genes comprises BRCA1, PMS2, RAD51, RAD54B, and RAD54L, wherein detecting the presence of at least one germline mutation in at least one of BRCA1, PMS2, RAD51, RAD54B, or RAD54L characterizes the prostate cancer in the subject as being an aggressive form of prostate cancer or as having an increased risk of developing into an aggressive form of prostate cancer. In some embodiments of all aspects of the present disclosure, there is a method of characterizing prostate cancer in a patient comprising assaying a biological sample obtained from the patient to determine if the biological sample contains at least one germline mutation in a plurality of human genes, wherein the plurality of human genes comprises: at least 15, such as at least 20, at least 25, at least 30, at least 35, at least 40, at least 45, or all 47 genes of the 47-gene panel; at least 13, such as at least 15, at least 20, or all 25 genes of the 25-gene panel; at least 10, such as at least 15, or all 19 genes of the 19-gene panel; at least 9, such as at least 10, at least 15, or all 16 genes of the 16-gene panel; at least 8, such as at least 10, or all 13 genes of the 13-gene panel; or at least 6, such as at least 7, or all 8 genes of the 8-gene panel, wherein detecting the presence of at least one germline mutation in at least one of the at least 15 human genes characterizes the prostate cancer in the subject as being an aggressive form of prostate cancer or as having an increased risk of developing into an aggressive form of prostate cancer. In certain embodiments, the patient is of African descent.

### Patient Treatment

Disclosed herein are methods for analyzing a biological sample from a patient for the presence or absence of germline mutations to obtain a specific gene mutation profile and using that gene mutation profile to predict and prognose prostate cancer. If a sample contains a mutation of certain genes, then there is an increased likelihood that the patient will develop prostate cancer in the future, has prostate cancer, and/or will have a worse prognosis than if the sample does not contain a mutation of any of the genes in the gene panel. As such, the methods of predicting, diagnosing or prognosing prostate cancer may also be used to assess the need for therapy or to monitor a response to a therapy (e.g., disease-free recurrence following surgery or other therapy). In the event of such a result, the methods of predicting the development of prostate cancer in the future may include one or more of the following steps: informing the patient that they are at an increased likelihood of developing cancer in the future; increasing the frequency of monitoring the subject for the development of prostate cancer or a more aggressive form of prostate cancer, and/or providing a prophylactic cancer treatment. The methods of prognosing cancer may include one or more of the following steps: informing the patient that they are likely to have prostate cancer; and treating the patient by an appropriate cancer therapy.

Also provided herein are methods of treating prostate cancer in a patient, such as a patient of African descent, the method comprising administering a prostate cancer treatment regimen to the patient, wherein prior to the administering step, the patient has been identified as having prostate cancer or a more advanced/aggressive form (e.g., poorly-differentiated prostate cancer) of prostate cancer. As discussed above, the presence of a mutation in a DDRG may increase a patient’s risk for developing cancer. Many DDRG mutations, however, confer an enhanced lethal response to therapies that induce DNA damage and/or apoptosis, thereby enhancing the sensitivity of cancer cells with DDRG mutations to such therapies. Such DNA damage control system therapies may include, for example, radiation, poly(ADP ribose) polymerase (PARP) inhibitors, and platinum-based therapeutics, as discussed below. Therefore, in certain embodiments, the methods disclosed herein may stratify patients, such as patients of African descent, by the mutation status for DNA damage control system therapies.

Prostate cancer treatment options include, but are not limited to, surgery, radiation therapy, hormone therapy, chemotherapy, biological therapy, or high intensity focused ultrasound. Drugs for prostate cancer treatment include, but are not limited to: Abiraterone Acetate, Cabazitaxel, Degarelix, Enzalutamide (XTANDI), Jevtana (Cabazitaxel), Prednisone, Provenge (Sipuleucel-T), Sipuleucel-T, or Docetaxel.

Additional drugs that may be used to treat prostate cancer include poly(ADP ribose) polymerase (PARP) inhibitors, immune checkpoint inhibitors, and platinum-based agents. PARP inhibitors may include, for example, olaparib, rucaparib, and niraparib. PARP1 is a protein that functions to repair single-stranded nicks in DNA. Drugs that inhibit PARP1 (PARP inhibitors) result in DNA containing multiple double stranded breaks during replication, which can lead to cell death. Immune checkpoint inhibitors work by blocking certain checkpoint proteins from binding with their partner proteins, allowing T cells to kill cancer cells. Immune checkpoint inhibitors may include, for example, pembrolizumab, nivolumab, and cemiplimab. Platinum-based agents are chemical complexes comprising platinum and cause crosslinking of DNA. Crosslinked DNA inhibits DNA repair and synthesis in cancerous cells. Exemplary platinum-based agents may include cisplatin, oxaliplatin, and carboplatin. Known DDRGs that are sensitive to PARP inhibitors or immune checkpoint inhibitors, for example, may include the genes in the 5-gene panel, the 16-gene panel, and the 8-gene panel. Accordingly, in certain embodiments of the methods disclosed herein, after detecting the presence of a germline mutation in at least one gene of the 5-gene panel, the 16-gene panel or the 8-gene panel, a patient who has been diagnosed with prostate cancer is treated by administration of a PARP inhibitor and/or an immune checkpoint inhibitor. In certain embodiments, the patient is of African descent.

A method as described in this application may, after a positive result, include a further therapy step, e.g., surgery, radiation therapy, hormone therapy, chemotherapy, biological therapy, or high intensity focused ultrasound. In certain embodiments, the therapy step comprises administering a DNA damage control system therapy, such as radiation, a PARP inhibitor, or a platinum-based agent.

### Compositions and Kits

The polynucleotide probes and/or primers or antibodies or polypeptide probes that can be used in the methods described herein can be arranged in a kit. Thus, one embodiment is directed to a kit for predicting, diagnosing, or prognosing prostate cancer comprising a plurality of polynucleotide probes for detecting a germline mutation in at least 1, such as at least 2, at least 3, at least 4, or 5 of the genes in the 5-gene panel, wherein the plurality of polynucleotide probes contains polynucleotide probes for no more than 500, 250, 100, 75, 60, 50, 47, 45, 40, 35, 30, 25, 20, 19, 16, 15, 13, 10, 9, 8, 6, or 5 genes.

Another embodiment is directed to a kit for predicting, diagnosing, or prognosing prostate cancer comprising a plurality of polynucleotide probes for detecting a germline mutation in at least 2, such as at least 5, at least 6, at least 8, at least 9, at least 10, at least 13, at least 15, at least 16, at least 19, at least 20, at least 25, at least 30, at least 35, at least 40, at least 45, or 47 of the genes in the 47-gene panel, wherein the plurality of polynucleotide probes contains polynucleotide probes for no more than 500, 250, 100, 75, 60, 50, 47, 45, 40, 35, 30, 25, 20, 19, 16, 15, 13, 10, 9, 8, 6, or 5 genes. In one embodiment, the plurality of polynucleotide probes comprises polynucleotide probes for detecting all 47 of the aforementioned genes.

In yet another embodiment, there is provided a kit for predicting, diagnosing, or prognosing cancer comprising a plurality of polynucleotide probes for detecting a germline mutation in at least 2, such as at least 5, at least 6, at least 8, at least 9, at least 10, at least 13, at least 15, at least 16, at least 19, at least 20, or 25 of the genes in the 25-gene panel, wherein the plurality of polynucleotide probes contains polynucleotide probes for no more than 500, 250, 100, 75, 60, 50, 40, 35, 30, 25, 20, 19, 16, 15, 13, 10, 9, 8, 6, or 5 genes. In one embodiment, the plurality of polynucleotide probes comprises polynucleotide probes for detecting all 25 of the aforementioned genes.

In yet another embodiment, there is provided a kit for predicting, diagnosing, or prognosing cancer comprising a plurality of polynucleotide probes for detecting a germline mutation in at least 2, such as at least 5, at least 6, at least 8, at least 9, at least 10, at least 13, at least 15, at least 16, or 19 of the genes in the 19-gene panel, wherein the plurality of polynucleotide probes contains polynucleotide probes for no more than 500, 250, 100, 75, 60, 50, 40, 35, 30, 25, 20, 19, 16, 15, 13, 10, 9, 8, 6, or 5 genes. In one embodiment, the plurality of polynucleotide probes comprises polynucleotide probes for detecting all 19 of the aforementioned genes.

In yet another embodiment, there is provided a kit for predicting, diagnosing, or prognosing cancer comprising a plurality of polynucleotide probes for detecting a germline mutation in at least 2, such as at least 5, at least 6, at least 8, at least 9, at least 10, at least 13, at least 15, or 16 of the genes in the 16-gene panel, wherein the plurality of polynucleotide probes contains polynucleotide probes for no more than 500, 250, 100, 75, 60, 50, 40, 35, 30, 25, 20, 19, 16, 15, 13, 10, 9, 8, 6, or 5 genes. In one embodiment, the plurality of polynucleotide probes comprises polynucleotide probes for detecting all 16 of the aforementioned genes.

In yet another embodiment, there is provided a kit for predicting, diagnosing, or prognosing cancer comprising a plurality of polynucleotide probes for detecting a germline mutation in at least 2, such as at least 5, at least 6, at least 8, at least 9, at least 10, or 13 of the genes in the 13-gene panel, wherein the plurality of polynucleotide probes contains polynucleotide probes for no more than 500, 250, 100, 75, 60, 50, 40, 35, 30, 25, 20, 19, 16, 15, 13, 10, 9, 8, 6, or 5 genes. In one embodiment, the plurality of polynucleotide probes comprises polynucleotide probes for detecting all 13 of the aforementioned genes.

In yet another embodiment, there is provided a kit for predicting, diagnosing, or prognosing cancer comprising a plurality of polynucleotide probes for detecting a germline mutation in at least 2, such as at least 5, at least 6, at least 7, or 8 of the genes in the 8-gene panel, wherein the plurality of polynucleotide probes contains polynucleotide probes for no more than 500, 250, 100, 75, 60, 50, 40, 35, 30, 25, 20, 19, 16, 15, 13, 10, 9, 8, 7, 6, or 5 genes. In one embodiment, the plurality of polynucleotide probes comprises polynucleotide probes for detecting all 8 of the aforementioned genes.

In certain embodiments, the kit comprises at least one polynucleotide probe for detecting a germline mutation in MDC1.

In one embodiment, the kit comprises at least one polynucleotide probe for detecting the expression of a control gene. The polynucleotide probes may be optionally labeled.

The kit may optionally include polynucleotide primers for amplifying a portion of the mRNA transcripts from at least 1, such as at least 2, at least 3, at least 4, or 5 of the genes in the 5-gene panel. In one embodiment, the kit optionally includes polynucleotide primers for amplifying a portion of the mRNA transcripts from all 5 of the aforementioned genes. In one embodiment, the kit comprises polynucleotide primers for amplifying a portion of the mRNA transcripts from a control gene.

In another embodiment, the kit may optionally include polynucleotide primers for amplifying a portion of the mRNA transcripts from at least 2, such as at least 5, at least 6, at least 8, at least 9, at least 10, at least 13, at least 15, at least 16, at least 19, at least 20, at least 25, at least 30, at least 35, at least 40, at least 45, or 47 of the genes in the 47-gene panel. In one embodiment, the kit optionally includes polynucleotide primers for amplifying a portion of the mRNA transcripts from all 47 of the aforementioned genes. In one embodiment, the kit comprises polynucleotide primers for amplifying a portion of the mRNA transcripts from a control gene.

In another embodiment, the kit optionally includes polynucleotide primers for amplifying a portion of the mRNA transcripts from at least 2, such as at least 5, at least 6, at least 8, at least 9, at least 10, at least 13, at least 15, at least 16, at least 19, at least 20, or 25 of the genes in the 25-gene panel.

In another embodiment, the kit optionally include polynucleotide primers for amplifying a portion of the mRNA transcripts from at least 2, such as at least 5, at least 6, at least 8, at least 9, at least 10, at least 13, at least 15, at least 16, or 19 of the genes in the 19-gene panel.

In another embodiment, the kit optionally includes polynucleotide primers for amplifying a portion of the mRNA transcripts from at least 2, at least 5, at least 6, at least 8, at least 9, at least 10, at least 13, at least 15, or 16 of the genes in the 16-gene panel.

In yet another embodiment, the kit optionally includes polynucleotide primers for amplifying a portion of the mRNA transcripts from at least 2, such as at least 5, at least 6, at least 8, at least 9, at least 10, or 13 of the genes in the 13-gene panel. In another embodiment, the kit optionally includes polynucleotide primers for amplifying a portion of the mRNA transcripts from at least 2, such as at least 5, at least 6, at least 7, or 8 of the genes in the 8-gene panel.

The kit for predicting, diagnosing, or prognosing prostate cancer may also comprise antibodies. Thus, in one embodiment, the kit for predicting, diagnosing, or prognosing prostate cancer comprises a plurality of antibodies for detecting at least 1, at least 2, at least 3, at least 4, or 5 of the polypeptides encoded by genes in the 5-gene panel, wherein the plurality of antibodies contains antibodies for detecting no more than 500, 250, 100, 75, 60, 50, 47, 45, 40, 35, 30, 25, 20, 19, 16, 15, 13, 10, 9, 8, 6, or 5 polypeptides.

In another embodiment, the kit for predicting, diagnosing, or prognosing prostate cancer comprises a plurality of antibodies for detecting at least 2, at least 5, at least 6, at least 8, at least 9, at least 10, at least 13, at least 15, at least 16, at least 19, at least 20, at least 25, at least 30, at least 35, at least 40, at least 45, or 47 of the polypeptides encoded by genes in the 47-gene panel, wherein the plurality of antibodies contains antibodies for detecting no more than 500, 250, 100, 75, 60, 50, 47, 45, 40, 35, 30, 25, 20, 19, 16, 15, 13, 10, 9, 8, 6, or 5 polypeptides.

In another embodiment, the kit for predicting, diagnosing, or prognosing prostate cancer comprises a plurality of antibodies for detecting at least 2, at least 5, at least 6, at least 8, at least 9, at least 10, at least 13, at least 15, at least 16, at least 19, at least 20, or 25 of the polypeptides encoded by genes in the 25-gene panel, wherein the plurality of antibodies contains antibodies for detecting no more than 500, 250, 100, 75, 60, 50, 40, 35, 30, 25, 20, 19, 16, 15, 13, 10, 9, 8, 6, or 5 polypeptides.

In another embodiment, the kit for predicting, diagnosing, or prognosing prostate cancer comprises a plurality of antibodies for detecting at least 2, at least 5, at least 6, at least 8, at least 9, at least 10, at least 13, at least 15, at least 16, or 19 of the polypeptides encoded by genes in the 19-gene panel, wherein the plurality of antibodies contains antibodies for detecting no more than 500, 250, 100, 75, 60, 50, 40, 35, 30, 25, 20, 19, 16, 15, 13, 10, 9, 8, 6, or 5 polypeptides.

In another embodiment, the kit for predicting, diagnosing, or prognosing prostate cancer comprises a plurality of antibodies for detecting at least 2, at least 5, at least 6, at least 8, at least 9, at least 10, at least 13, at least 15, or 16 of the polypeptides encoded by genes in the 16-gene panel, wherein the plurality of antibodies contains antibodies for detecting no more than 500, 250, 100, 75, 60, 50, 40, 35, 30, 25, 20, 19, 16, 15, 13, 10, 9, 8, 6, or 5 polypeptides. 13

In yet another embodiment, the kit for predicting, diagnosing, or prognosing prostate cancer comprises a plurality of antibodies for detecting at least 2, at least 5, at least 6, at least 7, or 8 of the polypeptides encoded by genes in the 8-gene panel, wherein the plurality of antibodies contains antibodies for detecting no more than 500, 250, 100, 75, 60, 50, 40, 35, 30, 25, 20, 19, 16, 15, 13, 10, 9, 8, 7, 6, or 5 polypeptides.. The antibodies may be optionally labeled.

As noted above, the polynucleotide or polypeptide probes and antibodies described herein may be optionally labeled with a detectable label. Any detectable label used in conjunction with probe or antibody technology, as known by one of ordinary skill in the art, can be used. As described herein, the labelled polynucleotide probes or labelled antibodies are not naturally occurring molecules; that is the combination of the polynucleotide probe coupled to the label or the antibody coupled to the label do not exist in nature. In certain embodiments, the probe or antibody is labeled with a detectable label selected from the group consisting of a fluorescent label, a chemiluminescent label, a quencher, a radioactive label, biotin, mass tags and/or gold.

In one embodiment, a kit includes instructional materials disclosing methods of use of the kit contents in a disclosed method. The instructional materials may be provided in any number of forms, including, but not limited to, written form (e.g., hardcopy paper, etc.), in an electronic form (e.g., computer diskette or compact disk) or may be visual (e.g., video files). The kits may also include additional components to facilitate the particular application for which the kit is designed. Thus, for example, the kits may additionally include other reagents routinely used for the practice of a particular method, including, but not limited to buffers, enzymes, labeling compounds, and the like. Such kits and appropriate contents are well known to those of skill in the art. The kit can also include a reference or control sample. The reference or control sample can be a biological sample or a data base.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

Unless indicated otherwise in these Examples, the methods involving commercial kits were done following the instructions of the manufacturers.

### Example 1

Whole genome sequencing (WGS) analysis was used to evaluate germline mutations in 276 DDRGs. The 276 analyzed DDRGs are listed below in Table 4. WGS was performed on 600 archived (CPDR Biospecimen Bank) blood DNA samples from African-American (n=300) and Caucasian American (n=300) patients who underwent radical prostatectomy primary treatment for prostate cancer at Walter Reed National Military Medical Center over the past 20 years. The samples were evaluated at the localized disease stage and not at metastasis.

The DNA samples were evaluated by both a Qubit® assay for quantity and Bioanalyzer® (Agilent Technologies) assay for quality, then diluted and aliquoted for WGS using the NovaSeq® (Illumina) platform. Of the 600 PCR-free libraries that were generated, 14 dropped out. Of the remaining 586 successful libraries, each was determined to have adequate quality based on DNA library metrics, including yield and fragment length. WGS depth exceeded 37x on average, and about 4 million single nucleotide polymorphisms (SNPs) were identified in the samples.

Patient genotypes were projected onto principle components from reference populations. Sample ancestries were predicted by the Peddy program, which uses a machine-learning model trained on individuals of diverse ancestries from the 1000 Genomes Project reference panel. Due to mismatched ancestry, 33 samples were excluded from further analysis. Furthermore, using the ContEst tool available from the Broad Institute’s Genome Analysis Toolkit (GATK), an additional 17 samples were excluded based on higher than minimal noise levels, such that n=259 for the African-American cohort and n=273 for the Caucasian-American cohort. No significant difference was found for any of the clinical variables between AA and CA men. To further determine population similarities between our cohort and reference control cohorts with common variants, we compared common variant allele frequencies (> 1%) and observed a high degree of correlation in AA individuals and in CA individuals (r²>0.99).

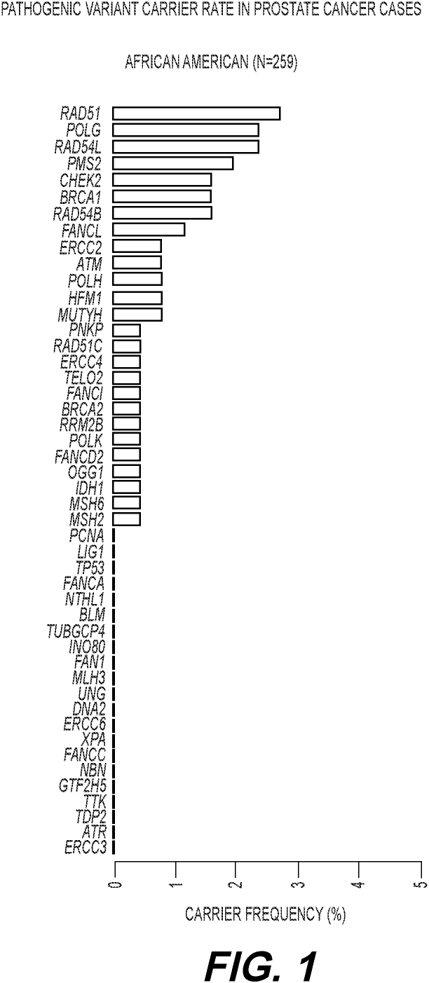

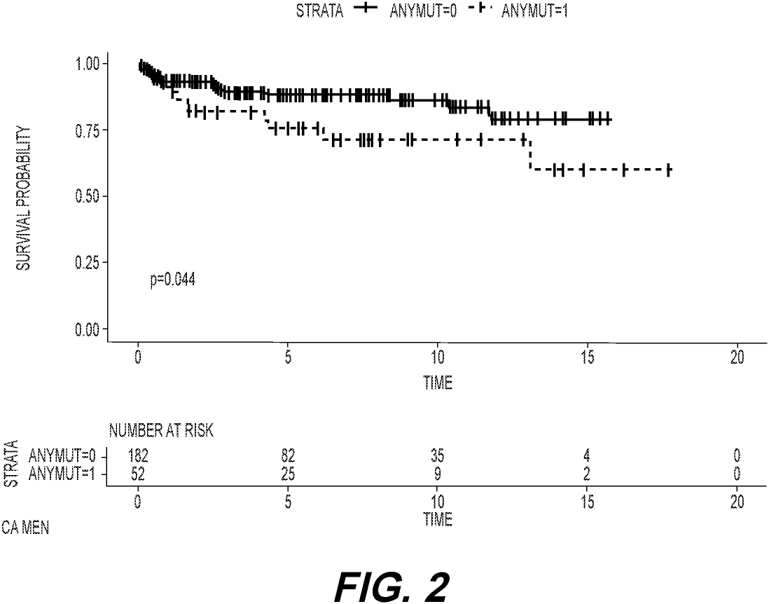

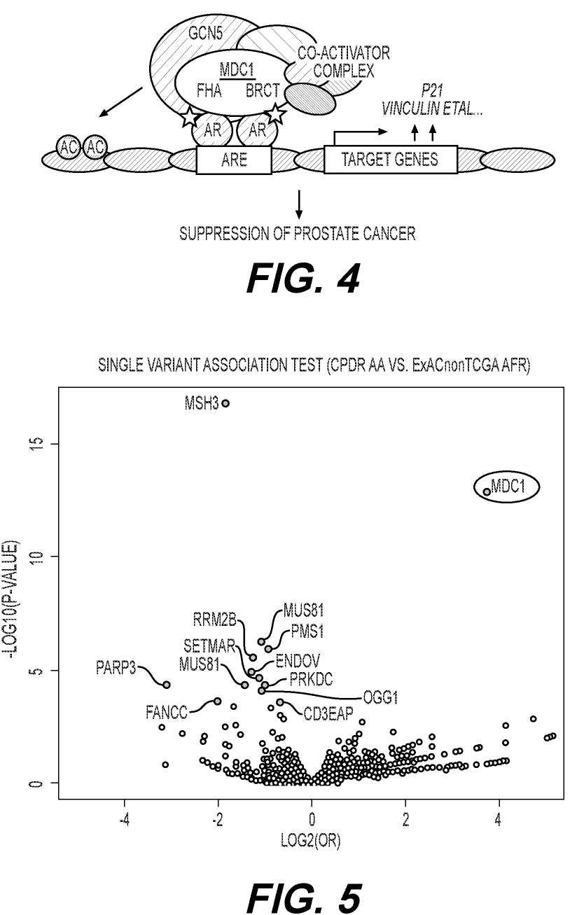

An inclusive set of DNA Damage Repair genes (276 genes; see Table 4) were evaluated for germline mutations that were predicted to have non-silent effects on the protein sequence (e.g., missense, nonsense, and frameshift mutations), as well as pathogenic/likely pathogenic (P/LP), and compared to publicly-available African American (n=1,890) and Caucasian American (n-18,740) controls from the Exome Aggregation Consortium (ExAC) (See FIGS. 3A and 3B). The single non-silent variant association between the CPDR samples of African-American men and the ExAc cohort of African-American men was plotted on a volcano plot. See FIG. 5. Several known and novel mutations were revealed. Forty-seven genes were determined to have at least one DDRG mutation, and 19 DDRGs were determined to have a mutation carrier frequency percent of 0.5% or greater, as shown below in Table 2. Eight DDRGs were determined to have a mutation carrier frequency percent of 1% or greater. See Table 2. The results are further illustrated graphically in FIG. 1.

TABLE 2

Pathogenic Carrier Rate in Prostate Cancer Cases

| DNA Damage Repair Gene | Carrier Frequency (%) | |
|---|---|---|
| | African-American patients (n=259) | Caucasian-American patients (n=273) |
| RAD51 | 2.70% (n=7) | — |
| RAD54L | 2.32% (n=6) | 0.73% (n=2) |
| POLG | 2.32% (n=6) | 4.40% (n=12) |
| PMS2 | 1.93% (n=5) | — |
| CHEK2 | 1.54% (n=4) | 1.10% (n=3) |
| RAD54B | 1.54% (n=4) | 0.37% (n=1) |
| BRCA1 | 1.54% (n=4) | — |
| FANCL | 1.16% (n=3) | 0.73% (n=2) |
| HFM1 | 0.77% (n=2) | — |
| ERCC2 | 0.77% (n=2) | 2.20% (n=6) |
| MUTYH | 0.77% (n=2) | 1.10% (n=3) |
| ATM | 0.77% (n=2) | 0.73% (n=2) |
| POLH | 0.77% (n=2) | — |
| BRCA2 | 0.39% (n=1) | — |
| TELO2 | 0.39% (n=1) | 0.37% (n=1) |
| FANCD2 | 0.39% (n=1) | 0.73% (n=2) |
| IDH1 | 0.39% (n=1) | 0.73% (n=2) |
| OGG1 | 0.39% (n=1) | 1.47% (n=4) |
| MSH6 | 0.39% (n=1) | 1.10% (n=3) |
| ERCC4 | 0.39% (n=1) | 0.37% (n=1) |
| RAD51C | 0.39% (n=1) | — |
| MSH2 | 0.39% (n=1) | — |
| FANC1 | 0.39% (n=1) | — |
| POLK | 0.39% (n=1) | — |
| RRM2B | 0.39% (n=1) | — |
| PNKP | 0.39% (n=1) | — |
| PCNA | — | 0.37% (n=1) |
| LIG1 | — | 0.37% (n=1) |
| TP53 | — | 0.37% (n=1) |
| FANCA | — | 2.93% (n=8) |
| NTHL1 | -— | 0.37% (n=1) |
| BLM | — | 0.37% (n=1) |
| FAN1 | — | 0.73% (n=2) |
| MLH3 | — | 0.37% (n=1) |
| UNG | — | 0.37% (n=1) |
| ERCC6 | — | 0.37% (n=1) |
| XPA | — | 0.37% (n=1) |
| FANCC | — | 0.37% (n=1) |
| NBN | — | 0.73% (n=2) |
| GTF2H5 | -— | 0.73% (n=2) |
| TDP2 | -— | 0.37% (n=1) |
| ATR | — | 0.37% (n=1) |
| ERCC2 | — | 0.37% (n=1) |
| TTK | — | 0.37% (n=1) |
| DNA2 | — | 0.37% (n=1) |
| INO80 | — | 0.37% (n=1) |
| TUBGCP4 | — | 0.73% (n=2) |
| MDC1 | 7.72% (n=20) | 2.56% (n=7) |

Of the 47 DDRG mutations detected, there were 16 DDRGs that are potentially targetable by therapeutics such as PARP inhibitors and/or immune checkpoint inhibitors. These 16 targetable DDRGs include BRCA1, BRCA2, FAN1, FANCA, FANCC, FANCD2, FANCI, FANCL, MLH, MSH2, MSH6, PMS2, RAD51, RAD51C, RAD54B, and RAD54L. Of those 16 targetable DDRGs, five were selected that had a high prevalence (over 1%) of germline mutations in the patients tested, particularly in patients of African American descent. These 5 targetable and prevalent DDRGs include BRCA1, PMS2, RAD51, RAD54B, and RAD54L. This 5-gene panel has a germline mutation in about 10% of the AA cohort tested (26 of 259) and in about 1.1% of the Caucasian cohort (3 of 272). The germline mutational frequencies of these 5 genes in the tested African American and Caucasian American cohorts are set forth in Table 3.

TABLE 3

Mutational Frequencies (MF) of Genes in 5-Gene Panel

| | RAD51 | RAD54L | RAD54B | BRAC1 | PMS2 |
|---|---|---|---|---|---|
| MF in AA Cohort | 2.7 % | 2.32% | 1.54% | 1.54% | 1.93% |
| MF in CA Cohort | 0% | 0.73% | 0.37% | 0% | 0% |

The 47 DDRG mutations were present in 125 patients out of the 531 patients evaluated (23.5%). Of these, 58 DDRG mutations were detected in the 259 African-American patient samples (22.8%) and 68 mutations were detected in the 272 Caucasian-American samples (24.3%). Compared to relevant literature, this study had the highest carrier rate in both AA men (22.8% vs. 10.1%; Nicolosi et al and 7.5%; Sartor et al) and in CA men (24.3% vs. 17.8%; Nicolosi et al and 13.9% Sartor et al). Nicolosi et al. identified eight DDRGs in the AA cohort (N=227) with a germline mutation. In our AA cohort (N=259), 23 DDRGs had a germline mutation (including seven of the eight genes reported in the Nicolosi et al. study). In the study by Sartor et al, 7.5% (16/214) AA men had pathogenic germline mutations in seven genes, of which six were identified in our analysis. Importantly, several of the most frequently mutated genes in AA CaP in our study were not tested by these two earlier studies. The most likely reason we observed three times higher percentage of patients with DDRG germline mutation compared to these two studies, is that we have tested for all DDRGs (276 genes).

Even though the percentage of patients found to have DDRG mutations (about 20%-25%) was similar for both African-American and Caucasian-American patients, 71.74% (33/46) of the identified 47 DDRGs with germline mutation were different across AA and CA patients, underscoring the racial disparity in DDRG germline mutations. See FIG. 1.

Although the analysis revealed that 47 of the 276 DDRGs were mutated, as shown in the FIG. 1, only the following 12 of the 40 mutated genes overlapped in both African-American and Caucasian-American patient samples: ATM, CHEK2, ERCC2, ERCC4, FANCD2, FANCL, MSH6, MUTYH, OGG1, POLG, RAD54B, and RAD54L.

As discussed above, 16 of the 47 identified DDRG mutations are in potentially targetable pathways. Testing for germline mutations in these 16 targetable DDRGs would have detected approximately 60% of the African-American patients having germline mutations (35 of 58) and approximately 32% of the Caucasian-American patients having germline mutations (22 of 68). Similarly, when looking at the eight identified targetable DDRGs having a mutation frequency greater than 1%, the racial distribution is uneven. 30 African-American patients are identified using the 8-gene panel, as compared to 11 Caucasian-American patients.

Several RAD family genes (RAD51, RAD54L, RAD54B), as well as PMS2 and BRCA1, were among the most frequently mutated DDRGs in AA patients, but not in CA patients, when compared to the relevant control datasets (FIG. 1). This is consistent with our previous study where we found that germline variants in BRCA1/2 were more frequent in AA than in CA CaP patients. Petrovics, G. et al., Prostate Cancer Prostatic Dis, 22: 406, 2019. Grouping all RAD mutations together from our DDRG gene set, we observed a greater rate in AA (6.95%) than in CA (1.10%) (P = 0.001). RAD51, RAD54L, RAD54B, PMS2, and BRCA1 are part of targetable DDRG pathways, specifically, the homologous recombination and mismatch repair pathways, which are known to respond to PARP inhibitor and immune checkpoint inhibitor therapy, respectively. Closer evaluation of the germline mutations identified that the mutations in RAD51 and PMS2 genes were enriched in AA compared to CA CaP patients, with p values of 0.0621 and 0.0268, respectively. On the other hand, FANCA was significantly more frequently mutated in CA compared to AA patients (p=0.0076) (FIG. 1). Twelve genes were recurrently mutated with 10 of them common to AA and CA patients (FANCA, MUTYH, OGG1, MSH6, POLG, RAD51, FANCL, RAD54L, CHEK2, POLH, NBN and TELO2). Five of these genes are part of targetable DDRG pathways (FANCA, MSH6, RAD51, FANCL, RAD54L) indicating a pathway for clinical intervention.

TABLE 4

DNA Damage Repair Genes

| HGNC Symbol | HGNC ID | Entrez gene No. | Description |
|---|---|---|---|
| APLF | 28724 | 200558 | aprataxin and PNKP like factor |
| APTX | 15984 | 54840 | aprataxin |
| ASCC3 | 18697 | 10973 | activating signal cointegrator 1 complex subunit 3 |
| DNTT | 2983 | 1791 | DNA nucleotidylexotransferase |
| LIG1 | 6598 | 3978 | DNA ligase 1 |
| LIG3 | 6600 | 3980 | DNA ligase 3 |
| LIG4 | 6601 | 3981 | DNA ligase 4 |
| MRE11A | 7230 | 4361 | MRE11 homolog A, double strand break repair nuclease |
| NBN | 7652 | 4683 | nibrin |
| NHEJ1 | 25737 | 79840 | nonhomologous end-joining factor 1 |
| PARG | 8605 | 8505 | poly(ADP-ribose) glycohydrolase |
| PARP1 | 270 | 142 | poly (ADP-ribose) polymerase 1 |
| PARP3 | 273 | 10039 | poly (ADP-ribose) polymerase family member 3 |
| PARPBP | 26074 | 55010 | PARP1 binding protein |
| PNKP | 9154 | 11284 | polynucleotide kinase 3′-phosphatase |
| POLB | 9174 | 5423 | polymerase (DNA directed), beta |
| POLL | 9184 | 27343 | polymerase (DNA directed), lambda |
| POLM | 9185 | 27434 | polymerase (DNA) mu |
| PRKDC | 9413 | 5591 | protein kinase, DNA-activated, catalytic polypeptid |
| RAD50 | 9816 | 10111 | RAD50 double strand break repair protein |
| RNF168 | 26661 | 165918 | ring finger protein 168 |
| RNF8 | 10071 | 9025 | ring finger protein 8 |
| TP53BP1 | 11999 | 7158 | tumor protein p53 binding protein 1 |
| XRCC1 | 12828 | 7515 | X-ray repair cross complementing 1 [Homo sapiens |
| XRCC2 | 12829 | 7516 | X-ray repair cross complementing 2 |
| XRCC3 | 12830 | 7517 | X-ray repair cross complementing 3 |
| XRCC4 | 12831 | 7518 | X-ray repair cross complementing 4 |
| XRCC5 | 12833 | 7520 | X-ray repair cross complementing 5 |
| XRCC6 | 4055 | 2547 | X-ray repair cross complementing 6 |
| UBE2A | 12472 | 7319 | ubiquitin-conjugating enzyme E2A |
| EXO1 | 3511 | 9156 | exonuclease 1 |
| HMGB1 | 4983 | 3146 | high mobility group box 1 |
| MLH1 | 7127 | 4292 | mutL homolog 1 |
| MLH3 | 7128 | 27030 | mutL homolog 3 |
| MSH2 | 7325 | 4436 | mutS homolog 2 |
| MSH3 | 7326 | 4437 | mutS homolog 3 |
| MSH6 | 7329 | 2956 | mutS homolog 6 |
| PCNA | 8729 | 5111 | proliferating cell nuclear antigen |
| PMS1 | 9121 | 5378 | PMS1 homolog 1, mismatch repair system component |
| PMS2 | 9122 | 5395 | PMS1 homolog 2, mismatch repair system component |
| POLD1 | 9175 | 5424 | polymerase (DNA directed), delta 1, catalytic subunit |
| POLD2 | 9176 | 5425 | polymerase (DNA directed), delta 2, accessory subunit |
| POLD3 | 20932 | 10714 | polymerase (DNA-directed), delta 3, accessory subunit |
| POLD4 | 14106 | 57804 | polymerase (DNA-directed), delta 4, accessory subunit |
| RFC1 | 9969 | 5981 | replication factor C subunit 1 |
| RFC2 | 9970 | 5982 | replication factor C subunit 2 |
| RFC3 | 9971 | 5983 | replication factor C subunit 3 |
| RFC4 | 9972 | 5984 | replication factor C subunit 4 |
| RFC5 | 9973 | 5985 | replication factor C subunit 5 |
| RPA1 | 10289 | 6117 | replication protein A1 |
| RPA2 | 10290 | 6118 | replication protein A2 |
| RPA3 | 10291 | 6119 | replication protein A3 |
| RPA4 | 30305 | 29935 | replication protein A4 |
| ALKBH1 | 17911 | 8846 | alkB homolog 1, histone H2A dioxygenase |
| ALKBH2 | 32487 | 121642 | alkB homolog 2, alpha-ketoglutarate dependent dioxygenase |
| ALKBH3 | 30141 | 221120 | alkB homolog 3, alpha-ketoglutarate dependent dioxygenase |
| APEX1 | 587 | 328 | apurinic/apyrimidinic endodeoxyribonuclease 1 |
| APEX2 | 17889 | 27301 | apurinic/apyrimidinic endodeoxyribonuclease 2 |
| APITD 1 | 23163 | 378708 | apoptosis-inducing, TAF9-like domain 1 |
| ATM | 795 | 472 | ATM serine/threonine kinase |
| ATR | 882 | 545 | ATR serine/threonine kinase |
| ATRIP | 33499 | 84126 | ATR interacting protein |
| ATRX | 886 | 546 | ATRX, chromatin remodeler |
| BARD1 | 952 | 580 | BRCA1 associated RING domain 1 |
| BLM | 1058 | 641 | Bloom syndrome, RecQ helicase-like |
| BRCA1 | 1100 | 672 | breast cancer 1, early onset |
| BRCA2 | 1101 | 675 | breast cancer 2, early onset |
| BRE | 1106 | 9577 | brain and reproductive organ-expressed (TNFRSF1A modulator) |
| BRIP1 | 20473 | 83990 | BRCA1 interacting protein C-terminal helicase 1 |
| CCNH | 1594 | 902 | cyclin H |
| CDK7 | 1778 | 1022 | cyclin-dependent kinase 7 |
| CETN2 | 1867 | 1069 | centrin, EF-hand protein, 2 |
| CHAF1A | 1910 | 10036 | chromatin assembly factor 1 subunit A |
| CHEK1 | 1925 | 1111 | checkpoint kinase 1 |
| CHEK2 | 16627 | 11200 | checkpoint kinase 2 |
| CLK2 | 2069 | 1196 | CDC-like kinase 2 |
| CUL3 | 2553 | 8452 | cullin 3 |
| CUL4A | 2554 | 8451 | cullin 4A |
| CUL5 | 2556 | 8065 | cullin 5 |
| DCLRE1 A | 17660 | 9937 | DNA cross-link repair 1A |
| DCLRE1 B | 17641 | 64858 | DNA cross-link repair 1B |
| DCLRE1 C | 17642 | 64421 | DNA cross-link repair 1C |
| DDB1 | 2717 | 1642 | damage specific DNA binding protein 1 |
| DDB2 | 2718 | 1643 | damage-specific DNA binding protein 2 |
| DMC1 | 2927 | 11144 | DNA meiotic recombinase 1 |
| DNA2 | 2939 | 1763 | DNA replication helicase/nuclease 2 |
| DUT | 3078 | 1854 | deoxyuridine triphosphatase |
| EID3 | 32961 | 493861 | EP300 interacting inhibitor of differentiation 3 |
| EME1 | 24965 | 146956 | essential meiotic structure-specific endonuclease 1 |
| EME2 | 27289 | 197342 | essential meiotic structure-specific endonuclease subunit 2 |
| ERCC1 | 3433 | 2067 | excision repair cross-complementation group 1 |
| ERCC2 | 3434 | 2068 | excision repair cross-complementation group 2 |
| ERCC3 | 3435 | 2071 | excision repair cross-complementation group 3 |
| ERCC4 | 3436 | 2072 | excision repair cross-complementation group 4 |
| ERCC5 | 3437 | 2073 | excision repair cross-complementation group 5 |
| ERCC6 | 3438 | 2074 | excision repair cross-complementation group 6 |
| ERCC8 | 3439 | 1161 | excision repair cross-complementation group 8 |
| FAAP10 0 | 26171 | 80233 | Fanconi anemia core complex associated protein 100 |
| FAAP24 | 28467 | 91442 | Fanconi anemia core complex associated protein 24 |
| FAAP20 | 26428 | 199990 | Fanconi anemia core complex associated protein 20 |
| FAM175 A | 25829 | 84142 | family with sequence similarity 175 |
| FAN1 | 29170 | 22909 | FANCD2/FANCI-associated nuclease 1 |
| FANCA | 3582 | 2175 | Fanconi anemia, complementation group A |
| FANCB | 3583 | 2187 | Fanconi anemia, complementation group B |
| FANCC | 3584 | 2176 | Fanconi anemia, complementation group C |
| FANCD2 | 3585 | 2177 | Fanconi anemia, complementation group D2 |
| FANCE | 3586 | 2178 | Fanconi anemia, complementation group E |
| FANCF | 3587 | 2188 | Fanconi anemia, complementation group F |
| FANCG | 3588 | 2189 | Fanconi anemia, complementation group G |
| FANCI | 25568 | 55215 | Fanconi anemia, complementation group I |
| FANCL | 20748 | 55120 | Fanconi anemia, complementation group L |
| FANCM | 23168 | 57697 | Fanconi anemia, complementation group M |
| FEN1 | 3650 | 2237 | flap structure-specific endonuclease 1 |
| GADD45 A | 4095 | 1647 | growth arrest and DNA-damage-inducible, alpha |
| GADD45 G | 4097 | 10912 | growth arrest and DNA-damage-inducible, gamma |
| GEN1 | 26881 | 348654 | GEN1 Holliday junction 5′ flap endonuclease |
| GTF2H1 | 4655 | 2965 | general transcription factor IIH subunit 1 |
| GTF2H2 | 4656 | 2966 | general transcription factor IIH subunit 2 |
| GTF2H3 | 4657 | 2967 | general transcription factor IIH subunit 3 |
| GTF2H4 | 4658 | 2968 | general transcription factor IIH subunit 4 |
| GTF2H5 | 21157 | 404672 | general transcription factor IIH subunit 5 |
| H2AFX | 4739 | 3014 | H2A histone family, member X |
| HELQ | 18536 | 113510 | helicase, POLQ-like |
| HES1 | 5192 | 3280 | hes family bHLH transcription factor 1 |
| HFM1 | 20193 | 164045 | HFM1, ATP dependent DNA helicase homolog |
| HLTF | 11099 | 6596 | helicase-like transcription factor |
| HMGB2 | 5000 | 3148 | high mobility group box 2 |
| HUS1 | 5309 | 3364 | HUS1 checkpoint clamp component |
| INO80 | 26956 | 54617 | catalytic ATPase subunit INO80 chromatin remodeling complex |
| KAT5 | 5275 | 10524 | K(lysine) acetyltransferase 5 |
| MAD2L2 | 6764 | 10459 | MAD2 mitotic arrest deficient-like 2 (yeast) |
| MBD4 | 6919 | 8930 | methyl-CpG binding domain 4, DNA glycosylase |
| MDC1 | 21163 | 9656 | mediator of DNA-damage checkpoint 1 |
| MGMT | 7059 | 4255 | O-6-methylguanine-DNA methyltransferase |
| MMS19 | 13824 | 64210 | MMS19 homolog, cytosolic iron-sulfur assembly component |
| MNAT1 | 7181 | 4331 | MNAT CDK-activating kinase assembly factor 1 |
| MPG | 7211 | 4350 | N-methylpurine-DNA glycosylase |
| MPLKIP | 16002 | 136647 | M-phase specific PLK1 interacting protein |
| MRPL40 | 14491 | 64976 | mitochondrial ribosomal protein L40 |
| MUS81 | 29814 | 80198 | MUS81 structure-specific endonuclease subunit |
| MUTYH | 7527 | 4595 | mutY DNA glycosylase |
| NABP2 | 28412 | 79035 | nucleic acid binding protein |
| NEIL1 | 18448 | 79661 | nei like DNA glycosylase 1 |
| NEIL2 | 18956 | 252969 | nei like DNA glycosylase 2 |
| NEIL3 | 24573 | 55247 | nei like DNA glycosylase 3 |
| NFATC2 IP | 25906 | 84901 | nuclear factor of activated T-cell 2 interacting protein |
| NSMCE1 | 29897 | 197370 | NSE1 homolog, SMC5-SMC6 complex component |
| NSMCE2 | 26513 | 286053 | NSE2/MMS21 homolog, SMC5-SMC6 complex SUMO ligase |
| NSMCE3 | 7677 | 56160 | NSE3 homolog, SMC5-SMC6 complex component |
| NSMCE4 A | 25935 | 54780 | NSE4 homolog A, SMC5-SMC6 complex component |
| NTHL1 | 8028 | 4913 | nth-like DNA glycosylase 1 |
| NUDT1 | 8048 | 4521 | nudix (nucleoside diphosphate linked moiety X)-type motif 1 |
| NUDT15 | 23063 | 55270 | nudix hydrolase 15 |
| NUDT18 | 26194 | 79873 | nudix hydrolase 18 |
| RRM1 | 10451 | 6240 | ribonucleotide reductase catalytic subunit M1 |
| RRM2 | 10452 | 6241 | ribonucleotide reductase regulatory subunit M2 |
| OGG1 | 8125 | 4968 | 8-oxoguanine DNA glycosylase |
| PALB2 | 26144 | 79728 | partner and localizer of BRCA2 |
| PARP2 | 272 | 10038 | poly (ADP-ribose) polymerase 2 |
| PARP4 | 271 | 143 | poly (ADP-ribose) polymerase family member 4 |
| PAXIP1 | 8624 | 22976 | PAX interacting protein 1 |
| PER1 | 8845 | 5187 | period circadian clock 1 |
| POLA1 | 9173 | 5422 | polymerase (DNA) alpha 1, catalytic subunit |
| POLE | 9177 | 5426 | polymerase (DNA directed), epsilon, catalytic subunit |
| POLE2 | 9178 | 5427 | polymerase (DNA directed), epsilon 2, accessory subunit |
| POLE3 | 13546 | 54107 | polymerase (DNA directed), epsilon 3, accessory subunit |
| POLE4 | 18755 | 56655 | polymerase (DNA-directed), epsilon 4, accessory subunit |
| POLG | 9179 | 5428 | polymerase (DNA) gamma, catalytic subunit |
| POLH | 9181 | 5429 | polymerase (DNA directed), eta |
| POLI | 9182 | 11201 | polymerase (DNA) iota |
| POLK | 9183 | 51426 | polymerase (DNA directed) kappa |
| POLN | 18870 | 353497 | polymerase (DNA) nu |
| POLQ | 9186 | 10721 | polymerase (DNA) theta |
| PPP4C | 9319 | 5531 | protein phosphatase 4, catalytic subunit |
| PPP4R1 | 9320 | 9989 | protein phosphatase 4, regulatory subunit 1 |
| PPP4R2 | 18296 | 151987 | protein phosphatase 4, regulatory subunit 2 |
| PPP4R4 | 23788 | 57718 | protein phosphatase 4, regulatory subunit 4 |
| PRPF19 | 17896 | 27339 | pre-mRNA processing factor 19 |
| RAD1 | 9806 | 5810 | RAD1 checkpoint DNA exonuclease |
| RAD17 | 9807 | 5884 | RAD17 checkpoint clamp loader component |
| RAD18 | 18278 | 56852 | RAD18 E3 ubiquitin protein ligase |
| RAD23A | 9812 | 5886 | RAD23 homolog A, nucleotide excision repair protein |
| RAD23B | 9813 | 5887 | RAD23 homolog B, nucleotide excision repair protein |
| RAD51 | 9817 | 5888 | RAD51 recombinase |
| RAD51B | 9822 | 5890 | RAD51 paralog B |
| RAD51C | 9820 | 5889 | RAD51 paralog C |
| RAD51D | 9823 | 5892 | RAD51 paralog D |
| RAD52 | 9824 | 5893 | RAD52 homolog, DNA repair protein |
| RAD54B | 17228 | 25788 | RAD54 homolog B (S. cerevisiae) |
| RAD54L | 9826 | 8438 | RAD54-like (S. cerevisiae) |
| RAD9A | 9827 | 5883 | RAD9 checkpoint clamp component A |
| RBBP8 | 9891 | 5932 | retinoblastoma binding protein 8 |
| RBX1 | 9928 | 9978 | ring-box 1 |
| RDM1 | 19950 | 201299 | RAD52 motif containing 1 |
| RECQL | 9948 | 5965 | RecQ helicase-like |
| RECQL4 | 9949 | 9401 | RecQ protein-like 4 |
| RECQL5 | 9950 | 9400 | RecQ protein-like 5 |
| REV1 | 14060 | 51455 | REV1, DNA directed polymerase |
| REV3L | 9968 | 5980 | REV3 like, DNA directed polymerase zeta catalytic subunit |
| RIF1 | 23207 | 55183 | replication timing regulatory factor 1 |
| RMI1 | 25764 | 80010 | BLAP75; FAAP75; C9orf76 |
| RMI2 | 28349 | 116028 | RecQ mediated genome instability 2 |
| RNMT | 10075 | 8731 | RNA guanine-7 methyltransferase |
| RRM2B | 17296 | 50484 | ribonucleotide reductase regulatory TP53 inducible subunit M2B |
| RTEL1 | 15888 | 51750 | regulator of telomere elongation helicase 1 |
| SETMA R | 10762 | 6419 | SET domain and mariner transposase fusion gene |
| SHFM1 | 10845 | 7979 | split hand/foot malformation (ectrodactyly) type 1 |
| SHPRH | 19336 | 257218 | SNF2 histone linker PHD RING helicase |
| SLX1A | 20922 | 548593 | SLX1 homolog A, structure-specific endonuclease subunit |
| SLX1B | 28748 | 79008 | GIYD2 |
| SLX4 | 23845 | 84464 | FANCP; BTBD12 |
| SMARC AD1 | 18398 | 56916 | SWI/SNF-related, regulator of chromatin, containing DEAD/H box 1 |
| SMC5 | 20465 | 23137 | structural maintenance of chromosomes 5 |
| SMC6 | 20466 | 79677 | structural maintenance of chromosomes 6 |
| SMUG1 | 17148 | 23583 | single-strand-selective monofunctional uracil-DNA glycosylase 1 |
| SPO11 | 11250 | 23626 | SPO11, initiator of meiotic double stranded breaks |
| STRA13 | 11422 | 201254 | stimulated by retinoic acid 13 |
| SWSAP1 | 26638 | 126074 | SWIM-type zinc finger 7 associated protein 1 |
| TCEA1 | 11612 | 6917 | transcription elongation factor A1 |
| TCEB1 | 11617 | 6921 | transcription elongation factor B subunit 1 |
| TCEB2 | 11619 | 6923 | transcription elongation factor B subunit 2 |
| TCEB3 | 11620 | 6924 | transcription elongation factor B subunit 3 |
| TDG | 11700 | 6996 | thymine-DNA glycosylase |
| TDP1 | 18884 | 55775 | tyrosyl-DNA phosphodiesterase 1 |
| TELO2 | 29099 | 9894 | telomere maintenance 2 |
| TOP3A | 11992 | 7156 | topoisomerase (DNA) III alpha |
| TOP3B | 11993 | 8940 | topoisomerase (DNA) III beta |
| TOPBP1 | 17008 | 11073 | topoisomerase (DNA) II binding protein 1 |
| TP53 | 11998 | 7157 | tumor protein p53 |
| TREX1 | 12269 | 11277 | three prime repair exonuclease 1 |
| TREX2 | 12270 | 11219 | three prime repair exonuclease 2 |
| TYMS | 12441 | 7298 | thymidylate synthetase |
| UBE2B | 12473 | 7320 | ubiquitin-conjugating enzyme E2B |
| UBE2N | 12492 | 7334 | ubiquitin-conjugating enzyme E2N |
| UBE2T | 25009 | 29089 | ubiquitin-conjugating enzyme E2T |
| UBE2V2 | 12495 | 7336 | ubiquitin-conjugating enzyme E2 variant 2 |
| UIMC1 | 30298 | 51720 | ubiquitin interaction motif containing 1 |
| UNG | 12572 | 7374 | uracil-DNA glycosylase |
| USP1 | 12607 | 7398 | ubiquitin specific peptidase 1 |
| UVSSA | 29304 | 57654 | UV stimulated scaffold protein A |
| WDR48 | 30914 | 57599 | WD repeat domain 48 |
| WRN | 12791 | 7486 | Werner syndrome, RecQ helicase-like |
| XAB2 | 14089 | 56949 | XPA binding protein 2 |
| XPA | 12814 | 7507 | xeroderma pigmentosum, complementation group A |
| XPC | 12816 | 7508 | xeroderma pigmentosum, complementation group C |
| ZSWIM7 | 26993 | 125150 | zinc finger SWIM-type containing 7 |
| PTEN | 9588 | 5728 | phosphatase and tensin homolog |
| TDP2 | 17768 | 51567 | tyrosyl-DNA phosphodiesterase 2 |
| ENDOV | 26640 | 284131 | endonuclease V |
| SPRTN | 25356 | 83932 | SprT-like N-terminal domain |
| RNF4 | 10067 | 6047 | ring finger protein 4 |
| SMARC A4 | 11100 | 6597 | SWI/SNF related, matrix associated, actin dependent regulator of chromatin, subfamily a, member 4 |
| IDH1 | 5382 | 3417 | isocitrate dehydrogenase (NADP(+)) 1, cytosolic |
| SOX4 | 11200 | 6659 | SRY-box 4 |
| WEE1 | 12761 | 7465 | WEE1 G2 checkpoint kinase |
| RAD9B | 21700 | 144715 | RAD9 checkpoint clamp component B |
| AEN | 25722 | 64782 | apoptosis enhancing nuclease |
| PLK3 | 2154 | 1263 | polo like kinase 3 |
| EXO5 | 26115 | 64789 | exonuclease 5 |
| CDC5L | 1743 | 998 | cell division cycle 5 like |
| BCAS2 | 975 | 10286 | BCAS2, pre-mRNA processing factor |
| PLRG1 | 9089 | 5356 | pleiotropic regulator 1 |
| YWHAB | 12849 | 7529 | tyrosine 3-monooxygenase/tryptophan 5-monooxygenase activation protein beta |
| YWHAG | 12852 | 7532 | tyrosine 3-monooxygenase/tryptophan 5-monooxygenase activation protein gamma |
| YWHAE | HGNC: 12 851 | 7531 | tyrosine 3-monooxygenase/tryptophan 5-monooxygenase activation protein epsilon |
| CDC25A | HGNC:17 25 | 993 | cell division cycle 25A |
| CDC25B | HGNC: 17 26 | 994 | cell division cycle 25B |
| CDC25C | HGNC: 17 27 | 995 | cell division cycle 25C |
| BABAM 1 | HGNC:25 008 | 29086 | BRISC and BRCA1 A complex member 1; aliases NBA1; HSPC142; MERIT40; C19orf62 |
| BRCC3 | HGNC:24 185 | 79184 | BRCAl/BRCA2-containing complex subunit 3 |
| TTK | HGNC: 12 401 | 7272 | TTK protein kinase |
| SMARC C1 | HGNC:11 104 | 6599 | SWI3; SWI/SNF related, matrix associated, actin dependent regulator of chromatin subfamily c member 1 |
| SWI5 | HGNC:31 412 | 375757 | SWI5 homologous recombination repair protein |
| MORF4L 1 | HGNC: 16 989 | 10933 | mortality factor 4 like 1 |
| RNF169 | HGNC:26 961 | 254225 | ring finger protein 169 |
| HERC2 | HGNC:48 68 | 8924 | HECT and RLD domain containing E3 ubiquitin protein ligase 2 |

### Example 2 - Correlation of DNA Damage Repair Gene Mutations with Biochemical Recurrence

To determine whether the presence of certain DDRG mutations may identify a patient as having an increased risk of biochemical recurrence (BCR) of prostate cancer, the patient samples discussed in Example 1 were further analyzed. Patient characteristics were examined for the presence or absence of a BCR, as well as the time elapsed to the BCR event. As used herein, BCR is the measure of PSA rise that initiates hormonal ablations and/or chemotherapy treatment. A BCR event was defined as a post-radical prostatectomy serum PSA level greater than 0.2 ng/mL, measured no less than eight weeks after radical prostatectomy, followed by a successive, confirmatory PSA level greater than or equal to 0.2 ng/mL or the initiation of salvage radiation or hormonal therapy after a rising PSA level greater than or equal to 0.1 ng/mL. Patients who had an initial serum PSA greater than 0.2 ng/mL but no rise of PSA and no initiation of salvage therapy were classified into the non-BCR event category.

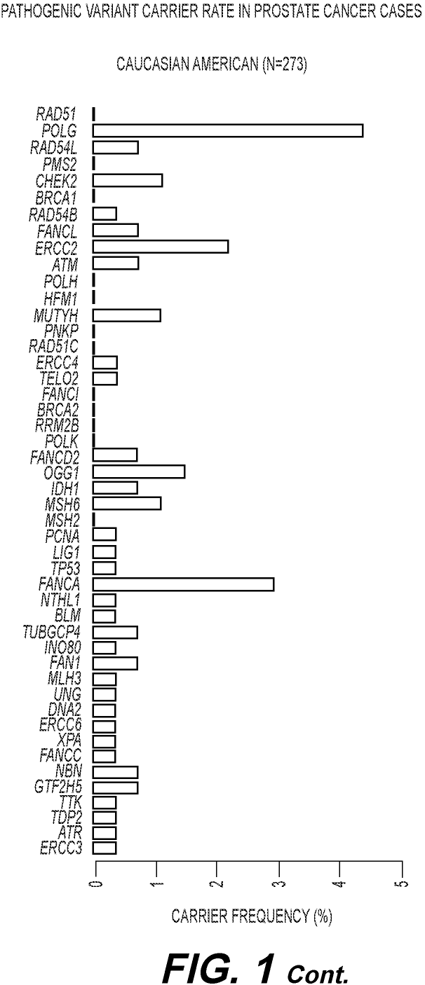

Cox proportional hazard modeling was used to evaluate the time to BCR, and a Kaplan-Meier plot (see FIG. 2) was used to visualize the effect. A germline mutation in any of the DDRG genes was associated with shorter time to BCR (Kaplan-Meier analysis, log rank p value 0.044) in AA patients, but not in CA patients (P = 0.74). In AA patients, an almost 2-fold higher percentage of BCR was found among patients with DDRG germline mutations (23.1%), compared to patients with no mutations (11.4%; P = 0.032), as shown in Table 3 below, while in CA men who underwent BCR, 12.7% of patients had germline mutation in comparison to 15.5% who carried no mutation (P = 0.59). A similar trend was observed for NCCN high risk category in AA men where significantly higher frequency of DDRG mutations was found (P = 0.021) compared to low and intermediate risk categories. No significant differences in distribution of DDRG mutations for clinical outcome was observed in CA men.

TABLE 3

Correlation between BCR and DDRG Mutations in African-American Patient Cohort

| BCR | No DDRG Mutation | At Least One DDRG Mutation | p-value |
|---|---|---|---|
| No | 87.7% (n=185) | 76.9% (n=40) | 0.048 |
| Yes | 12.3% (n=26) | 23.1 % (n=12) | |

In the African-American patients exhibiting at least one DDRG mutation, the following six DDRGs were found to be mutated only in patients having a BCR: BRCA2, ERCC2, FANCI, MSH6, OGG1, and RAD51C. The genes RAD51, RAD54L, and FANCL, however, were found to have mutations in both patients who had a BCR and those who did not.

The survival probability over 20 months was also decreased between African-American patients who had at least one DDRG germline mutation and African-American patients who did not (p=0.044), as shown in FIG. 2.

### Example 3 - Pathogenic Variant Validation With Droplet Digital PCR (ddPCR)

The mutations identified by WGS were further confirmed by using a Droplet Digital Polymerase Chain Reaction (ddPCR) technique using a QX200 Droplet Generator (BioRad). The data was analyzed by QuantaSoft software (BioRad). A ddPCR mastermix was prepared containing 11 µl 2X ddPCR Supermix (Bio-Rad), 1.1 µl 20X TaqMan SNP Genotyping Assay (Bio-Rad, Applied Biosystems), and 7.9 µl nuclease-free water (Qiagen) per sample. The mastermix was prepared at room temperature, and 20 µl was added to 2 µl (5 ng) of each DNA sample. Samples were loaded into individual wells of DG8TM cartridges (BioRad), and droplets were generated using a QX200 Droplet Generator (BioRad). For each sample, 40 µl of droplet mix was then transferred to a 96-well plate, and PCR was performed in a thermal cycler using the following cycling conditions: 95° C. x 10 min; 40 cycles of [94° C. x 30 s, 60° C. x 60 s]; 98° C. x 10 s; and 40° C. x 10 min. The Bio-Rad QX200 Droplet Reader was then used to assess droplets as positive or negative based on fluorescence amplitude. The QuantaSoft software (BioRad) was used to analyze droplet data. The ddPCR assay results agreed with WGS results in 99.15% (117 of 118) of cases. The results are shown below in Table 4.

TABLE 4

DDRG ddPCR Validation

| Chromosome | Location | Ref. allele | Alt. allele | Gene | Type | Mutation | Amino acid change | Validated? |
|---|---|---|---|---|---|---|---|---|
| 1 | 46260853 | C | T | RAD54L | Exonic | Missense | R202C | Yes |
| 2 | 47800616 | T | C | MSH6 | Exonic | Missense | V748A | Yes |
| 17 | 43106478 | A | C | BRCA1 | Exonic | Missense | C17G | Yes |
| 15 | 40731103 | C | T | RAD51 | Exonic | Missense | R275X | Yes |
| 15 | 89326947 | C | A | POLG | Exonic | Missense | G517V | Yes |
| 15 | 89330184 | G | A | POLG | Exonic | Missense | T251I | Yes |
| 13 | 32394814 | C | T | BRCA2 | Exonic | Missense | R3128X | Yes |
| 15 | 40718818 | G | A | RAD51 | Exonic | Missense | R151Q | Yes |
| 15 | 89323423 | A | G | POLG | Exonic | Missense | F749S | Yes |
| 15 | 89323460 | C | G | POLG | Exonic | Missense | G737R | Yes |
| 15 | 89330106 | T | A | POLG | Exonic | Missense | H277L | Yes |
| 15 | 89327166 | C | T | POLG | Splicing | Unknown | | Yes |
| 17 | 43104967 | A | C | BRCA1 | Intronic | Unknown | | Yes |
| 8 | 94391872 | C | T | RAD54B | Exonic | Missense | A332T | Yes |
| 1 | 45331556 | C | T | MUTYH | Exonic | Missense | G253D | Yes |
| 7 | 5997415 | G | T | PMS2 | Exonic | Missense | S132R | Yes |
| 7 | 5999165 | G | C | PMS2 | Exonic | Missense | C110W | Yes |
| 7 | 5999193 | C | T | PMS2 | Exonic | Missense | G101E | Yes |
| 8 | 94391698 | C | A | RAD54B | Exonic | Missense | G390W | Yes |
| 8 | 94393778 | G | A | RAD54B | Exonic | Missense | P311S | Yes |
| 8 | 94400440 | A | G | RAD54B | Exonic | Missense | I139T | Yes |
| 15 | 89325639 | G | A | POLG | Exonic | Missense | P587L | Yes for 1 patient; No for 1 patient* |
| 1 | 46260597 | C | T | RAD54L | Exonic | Missense | P155S | Yes |
| 1 | 46278086 | G | A | RAD54L | Exonic | Missense | R683H | Yes |
| 11 | 108281168 | G | A | ATM | Exonic | Silent | K1192K | Yes |
| 11 | 108304673 | A | C | ATM | Splicing | Unknown | | Yes |
| 11 | 108343221 | G | A | ATM | Splicing | Unknown | | Yes |
| 22 | 28712018 | C | T | CHEK2 | Splicing | Unknown | | Yes |
| 22 | 28725099 | A | G | CHEK2 | Exonic | Missense | I157T | Yes |
| 19 | 45352249 | G | C | ERCC2 | Exonic | Missense | A717G | Yes |
| 19 | 45352331 | G | A | ERCC2 | Exonic | Missense | R690W | Yes |
| 19 | 45364463 | G | A | ERCC2 | Exonic | Missense | R203C | Yes |
| 19 | 45365128 | C | G | ERCC2 | Exonic | Missense | D107H | Yes |
| 19 | 45368655 | C | T | ERCC2 | Exonic | Missense | R88H | Yes |
| 2 | 58159793 | G | GTAAT | FANCL | Exonic | Frame shift | T372fs | Yes |
| 15 | 89325639 | G | A | POLG | Exonic | Missense | P587L | No* |
| chr16 | 89767168 | G | C | FANCA | Exonic | Missense | S585R | Yes |
| chr13 | 32394814 | C | T | BRCA2 | Exonic | Missense | R3128X | Yes |
| chr15 | 30905606 | C | T | FAN1 | Exonic | Missense | Q315X | Yes |
| chr9 | 95125085 | C | A | FANCC | Splicing | Unknown | | Yes |
| chr14 | 75047174 | C | A | MLH3 | Exonic | Nonsense | E828X | Yes |
| chr17 | 58703194 | A | G | RAD51C | Splicing | Unknown | | Yes |
| chr2 | 47800616 | T | C | MSH6 | Exonic | Missense | V748A | Yes |

* One patient sample excluded due to quality control issue

In all, 42 of 43 mutations in 74 patients were validated by the ddPCR method. The total number of patients screened including controls (wt) was 119, with a 99.15% concordance (118/119).

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed:

1. A method of predicting a predisposition for developing prostate cancer in a patient, the method comprising:
assaying a biological sample obtained from the patient to determine if the biological sample contains at least one pathogenic or likely pathogenic gene mutation in a plurality of genes, wherein the plurality of genes comprises the 5 following human genes: BRCA1, PMS2, RAD51, RAD54B, and RAD54L, wherein the patient is identified as having a predisposition for developing prostate cancer if at least one pathogenic or likely pathogenic gene mutation is detected in BRCA1, PMS2, RAD51, RAD54B, or RAD54L.

2. A method of obtaining a gene mutation profile in a biological sample from a patient, the method comprising:
assaying a biological sample obtained from the patient to determine if the biological sample contains at least one pathogenic or likely pathogenic gene mutation in a plurality of genes, wherein the plurality of genes comprises the following human genes: BRCA1, PMS2, RAD51, RAD54B, and RAD54L.

3. A method of selecting a treatment for a patient with prostate cancer, the method comprising:
assaying a biological sample obtained from the patient to determine if the biological sample contains at least one pathogenic or likely pathogenic gene mutation in a plurality of genes, wherein the plurality of genes comprises the following human genes: BRCA1, PMS2, RAD51, RAD54B, and RAD54L,
selecting a treatment for the patient if at least one pathogenic or likely pathogenic gene mutation is detected in BRCA1, PMS2, RAD51, RAD54B, or RAD54L, wherein the selected treatment comprises surgery, radiation, hormone therapy, chemotherapy, biological therapy, targeted therapy, or high intensity focused ultrasound.

4. The method of claim 3, wherein the selected treatment comprises a therapy that induces DNA damage and/or apoptosis.

5. The method of claim 4, wherein the therapy that induces DNA damage and/or apoptosis comprises radiation, a poly(ADP ribose) polymerase (PARP) inhibitor, or a platinum-based therapeutic.

6. A method for stratifying prostate cancer in a patient, the method comprising:
assaying a biological sample obtained from the patient to determine if the biological sample contains at least one pathogenic or likely pathogenic gene mutation in a plurality of genes, wherein the plurality of genes comprises the following human genes BRCA1, PMS2, RAD51, RAD54B, and RAD54L,
stratifying the prostate cancer patients into different molecular subtypes and identifying those having an increased risk of biochemical recurrence following radical prostatectomy if at least one pathogenic or likely pathogenic gene mutation is detected in the plurality of genes.

7. The method of any of the preceding claims, wherein the plurality of genes further comprises at least 10 of the following 42 human genes: ATM, ATR, BLM, BRCA2, CHEK2, DNA2, ERCC2, ERCC3, ERCC4, ERCC6, FAN1, FANCA, FANCC, FANCD2, FANCI, FANCL, GTF2H5, HFM1, IDH1, INO80, LIG1, MLH3, MSH2, MSH6, MUTYH, NBN, NTHL1, OGG1, PCNA, PNKP, POLG, POLH, POLK, RAD51C, RRM2B, TDP2, TP53, TELO2, TTK, TUBGCP4, UNG, and XPA.

8. The method of any of the preceding claims, wherein the plurality of genes further comprises at least 8 of the following 20 human genes: ATM, BRCA2, CHEK2, ERCC2, FAN1, FANCA, FANCC, FANCD2, FANCI, FANCL, GTF2H5, MLH3, MSH2, MSH6, MUTYH, NBN, OGG1, POLG, POLH, and RAD51C.

9. The method of any one of claims 1-6, wherein the plurality of genes further comprises at least 5 of the following 14 human genes: ATM, CHEK2, ERCC2, FAN1, FANCA, FANCD2, FANCL, GTF2H5, MSH6, MUTYH, NBN, OGG1, POLG, and POLH.

10. The method of any one of claims 1-6, wherein the plurality of genes further comprises at least 4 of the following 11 human genes: BRCA2, FAN1, FANCA, FANCC, FANCD2, FANCI, FANCL, MLH, MSH2, MSH6, and RAD51 C.

11. The method of any one of claims 1-6, wherein the plurality of genes further comprises at least 3 of the following 8 human genes: CHEK2, ERCC2, FANCA, FANCL, MSH6, MUTYH, OGG1, and POLG.

12. The method of any one of claims 1-6, wherein the plurality of genes further comprises at least one of the following 3 human genes: FANCA, FANCL, and MSH6.

13. The method of claim 7, wherein the plurality of genes further comprises all 42 genes.

14. The method of claim 8, wherein the plurality of genes further comprises all 20 genes.

15. The method of claim 9, wherein the plurality of genes further comprises all 14 genes.

16. The method of claim 10, wherein the plurality of genes further comprises all 11 genes.

17. The method of claim 11, wherein the plurality of genes further comprises all 8 genes.

18. The method of claim 12, wherein the plurality of genes further comprises all 3 genes.

19. The method according to any of the preceding claims, wherein the biological sample is assayed using sequencing techniques.

20. The method according to claim 19, wherein each of the genes in the plurality of genes is sequenced before determining if the biological sample contains at least one pathogenic or likely pathogenic gene mutation in the plurality of genes.

21. The method of any one of claims 1-18, wherein nucleic acid expression is detected.

22. The method of any one of claims 1-18, wherein polypeptide expression is detected.

23. The method according to any of the preceding claims, wherein the patient is of African descent.

24. The method according to any of the preceding claims, wherein the biological sample comprises the patient’s blood or saliva or is obtained therefrom.

25. The method of any of the preceding claims, further comprising a step of providing genetic counseling to the patient.

26. The method of claim 25, wherein the patient has a family history of cancer.

27. The method of claim 26, wherein the family history of cancer comprises a family history of DDRG germline mutation related cancer.

28. The method of claim 27, wherein the DDRG germline mutation related cancer is breast cancer or prostate cancer.

29. The method of any of the preceding claims, wherein if at least one pathogenic or likely pathogenic gene mutation is detected in the plurality of genes, the method further comprises a step of treating the patient.

30. The method of claim 29, wherein treating the patient comprises surgery, radiation, hormone therapy, chemotherapy, biological therapy, targeted therapy, or high intensity focused ultrasound.

31. The method of claim 30, wherein treating the patient comprises administering a therapy that induces DNA damage and/or apoptosis.

32. The method of claim 31, wherein the therapy that induces DNA damage and/or apoptosis comprises radiation, a poly(ADP ribose) polymerase (PARP) inhibitor, or a platinum-based therapeutic.

33. A kit for use in predicting, diagnosing, and/or prognosing cancer, the kit comprising a plurality of probes for detecting a pathogenic or likely pathogenic gene mutation in at least the following human genes BRCA1, PMS2, RAD51, RAD54B, and RAD54L,
wherein the plurality of probes contains probes for detecting the pathogenic or likely pathogenic gene mutation in no more than 500 different genes.

34. The kit of claim 33, wherein the plurality of probes further comprises probes for detecting a pathogenic or likely pathogenic gene mutation in at least 10 of the following 42 human genes: ATM, ATR, BLM, BRCA2, CHEK2, DNA2, ERCC2, ERCC3, ERCC4, ERCC6, FAN1, FANCA, FANCC, FANCD2, FANCI, FANCL, GTF2H5, HFM1, IDH1, INO80, LIG1, MLH3, MSH2, MSH6, MUTYH, NBN, NTHL1, OGG1, PCNA, PNKP, POLG, POLH, POLK, RAD51C, RRM2B, TDP2, TP53, TELO2, TTK, TUBGCP4, UNG, and XPA.

35. The kit of claim 33, wherein the plurality of probes further comprises probes for detecting a pathogenic or likely pathogenic gene mutation in at least 8 of the following 20 human genes: ATM, BRCA2, CHEK2, ERCC2, FAN1, FANCA, FANCC, FANCD2, FANCI, FANCL, GTF2H5, MLH3, MSH2, MSH6, MUTYH, NBN, OGG1, POLG, POLH, and RAD51C.

36. The kit according to claim 33, wherein the plurality of probes further comprises probes for detecting a pathogenic or likely pathogenic gene mutation in at least 5 of the following 14 human genes: ATM, CHEK2, ERCC2, FAN1, FANCA, FANCD2, FANCL, GTF2H5, MSH6, MUTYH, NBN, OGG1, POLG, and POLH.

37. The kit of claim 33, wherein the plurality of probes further comprises probes for detecting a pathogenic or likely pathogenic gene mutation in at least 4 of the following 11 human genes: BRCA2, FAN1, FANCA, FANCC, FANCD2, FANCI, FANCL, MLH, MSH2, MSH6, and RAD51C.

38. The kit according to claim 33, wherein the plurality of probes further comprises probes for detecting a pathogenic or likely pathogenic gene mutation in at least 3 of the following 8 human genes: CHEK2, ERCC2, FANCA, FANCL, MSH6, MUTYH, OGG1, and POLG.

39. The kit according to claim 33, wherein the plurality of probes further comprises probes for detecting a pathogenic or likely pathogenic gene mutation in at least one of the following 3 human genes: FANCA, FANCL, and MSH6.

40. The kit of claim 34, wherein the plurality of probes contains probes for detecting a pathogenic or likely pathogenic gene mutation in all 42 genes.

41. The kit of claim 35, wherein the plurality of probes contains probes for detecting a pathogenic or likely pathogenic gene mutation in all 20 genes.

42. The kit of claim 36, wherein the plurality of probes contains probes for detecting a pathogenic or likely pathogenic gene mutation in all 14 genes.

43. The kit of claim 37, wherein the plurality of probes contains probes for detecting a pathogenic or likely pathogenic gene mutation in all 11 genes.

44. The kit of claim 38, wherein the plurality of probes contains probes for detecting a pathogenic gene mutation or likely pathogenic gene mutation in all 8 genes.

45. The kit of claim 39, wherein the plurality of probes contains probes for detecting a pathogenic or likely pathogenic gene mutation in all 3 genes.

46. The kit of any one of claims 33-45, wherein the plurality of probes is selected from a plurality of oligonucleotide probes, a plurality of antibodies, or a plurality of polypeptide probes.

47. The kit of any one of claims 33-46, wherein the plurality of probes contains probes for detecting a pathogenic or likely pathogenic gene mutation in no more than 250, 100, 75, 60, 50, 40, 30, 25, 20, 19, 16, 15, 13, 9, 10, 8, or 6 different genes.

48. The kit of any one of claims 33-47, wherein the plurality of probes is attached to the surface of an array.

49. The kit of claim 47, wherein the array comprises no more than 250, 100, 75, 60, 50, 40, 30, 25, 20, 19, 16, 15, 13, 9, 10, 8, or 6 different addressable elements.

50. The kit of any one of claims 33-49, wherein the plurality of probes is labeled.

51. A genetic testing method for identifying a patient having a predisposition for developing prostate cancer, the method comprising:
obtaining a biological sample from the patient;
assaying the biological sample to determine if the biological sample contains at least one pathogenic or likely pathogenic gene mutation in a plurality of genes, wherein the plurality of genes comprises the following human genes BRCA1, PMS2, RAD51, RAD54B, and RAD54L; and
identifying the patient as having a predisposition for developing prostate cancer if at least one pathogenic or likely pathogenic gene mutation is detected in at least one of BRCA1, PMS2, RAD51, RAD54B, or RAD54L.

52. The method of claim 51, wherein prior to assaying the biological sample, the patient has a family history of cancer.

53. The method of claim 52, wherein the family history of cancer is a family history of DDRG germline mutation related cancer.

54. The method of claim 53, wherein the DDRG germline mutation related cancer is breast cancer or prostate cancer.

55. The method of any one of claims 50-54, wherein the patient is of African descent.

56. A method of characterizing prostate cancer in a patient, the method comprising:
assaying a biological sample obtained from the patient to determine if the biological sample contains at least one pathogenic or likely pathogenic gene mutation in a plurality of genes, wherein the plurality of genes comprises the following human genes: BRCA1, PMS2, RAD51, RAD54B, and RAD54L;
wherein detecting the presence of at least one pathogenic or likely pathogenic gene mutation in at least one of BRCA1, PMS2, RAD51, RAD54B, or RAD54L characterizes the prostate cancer in the subject as being an aggressive form of prostate cancer or as having an increased risk of developing into an aggressive form of prostate cancer.

57. The method of claim 56, wherein the patient is of African descent.
